(19) United States
(12) Patent Application Publication
(10) Pub. No.: US 2003/0219763 A1
(43) Pub. Date: Nov. 27, 2003

(54) Plant protoplast gene expression systems and uses thereof

(76) Inventor: Jen Sheen, Boston, MA (US)

Correspondence Address: CLARK & ELBING LLP, 101 FEDERAL STREET, BOSTON, MA, 02110, US

(21) Appl. No.: 10/243,581

(22) Filed: Sep. 13, 2002

Related U.S. Application Data
(63) Continuation of application No. PCT/US01/07999, filed on Mar. 13, 2001.
(63) Continuation-in-part of application No. 08/989,881, filed on Dec. 12, 1997.
(63) Continuation-in-part of application No. 09/371,338, filed on Aug. 10, 1999, now Pat. No. 6,613,959.
(60) Provisional application No. 60/189,074, filed on Mar. 13, 2000.
(60) Provisional application No. 60/032,966, filed on Dec. 13, 1996.
(60) Provisional application No. 60/095,938, filed on Aug. 10, 1998.

(51) Int. Cl. C12Q 1/68

(52) U.S. Cl. 435/6

(57) ABSTRACT

Disclosed are high throughput assays for rapidly screening a library of nucleic acid molecules to identify a gene product that modulates expression of a gene of interest. The assays generally involve (a) introducing into one or more plant protoplasts (i) a reporter gene construct operably linked to a promoter of a gene of interest and (ii) a member of a library of nucleic acid molecules, wherein the library member is expressed in the plant protoplasts; and (b) screening the protoplasts to determine whether the amount of gene expression of the reporter gene construct changes in response to the expression of the library member, a change in gene expression of the reporter gene construct identifying the gene product expressed by the library member as one that modulates expression of the gene of interest.

### CROSS REFERENCE TO RELATED APPLICATIONS

This application claims benefit of PCT international application number US01/07999, filed on Mar. 13, 2001, which, in turn, claims benefit of U.S. provisional patent application No. 60/189,074, filed on Mar. 13, 2000, now abandoned, and is a continuation-in-part of U.S. patent application Ser. No. 08/989,881, filed on Dec. 12, 1997, which claims benefit of U.S. provisional application No. 60/032,966, filed on Dec. 13, 1996, now abandoned, and U.S. patent application Ser. No. 09/371,338, filed on Aug. 10, 1999, which claims benefit of U.S. provisional application No. 60/095,938, filed on Aug. 10, 1998, now abandoned.

### STATEMENT AS TO FEDERALLY SPONSORED RESEARCH

This invention was made in part with Government funding, and the Government therefore has certain rights in the invention.

### BACKGROUND OF THE INVENTION

The invention relates to functional analysis of gene expression using plant protoplasts.

Destined to reside in the habitats of germination, plants are frequently exposed to unfavorable environmental conditions. Extreme temperature, drought, salinity, pollution, and pathogens greatly affect plant growth, development, and productivity. To survive, plants have developed a complex signaling network that senses and protects them from an ever-changing environment. A common plant response to different abiotic and biotic stresses, such as heat, chilling, excessive light, drought, wounding, ozone exposure, UV-B irradiation, osmotic shock, and pathogens is the accelerated generation or/and accumulation of reactive oxygen species, including hydrogen peroxide (H₂O₂), superoxide anion and hydroxyl radicals. Genetic and biochemical analysis of plant signaling cascades is not straightforward because the key regulators are typically functionally redundant, expressed at low levels, or have indispensable roles for cell viability (Meissner et al., Plant Cell 11, 1827-1840. 1999). Accordingly, a need exists for alternative approaches to unravel the function of known regulatory genes, as well as to identify novel genes regulating plant signaling cascades.

### SUMMARY OF THE INVENTION

In one aspect, the invention features a method for determining whether a polypeptide modulates transcription of a gene of interest. The method, in general, involves: (a) introducing into one or more plant protoplasts (i) a reporter gene construct operably linked to a promoter of a gene of interest and (ii) an effector construct that expresses a polypeptide; and (b) determining whether the amount of gene expression of the reporter gene construct changes in response to the expression of the effector construct, a change in gene expression of the reporter gene construct identifying the polypeptide expressed by the effector construct as one that modulates transcription of the gene of interest. In preferred embodiments, the method includes treating the protoplasts (e.g., mesophyll protoplasts) with a biotic or an abiotic stress; a hormone; a metabolite; or with a pathogen, elicitor, or elicitin. In other preferred embodiments, the gene of interest expresses a signaling pathway polypeptide.

Preferably, the reporter gene and effector construct are introduced into the protoplasts by electroporation or polyethylene glycol transformation; and the plant protoplasts are maize or Arabidopsis protoplasts. In preferred embodiments, the method includes approximately 10⁴ to 10⁶ plant protoplasts. In still other embodiments, the reporter gene expresses a green fluorescent protein, a firefly luciferase, or a β-glucuronidase; the effector construct encodes a signaling pathway polypeptide.

In still other preferred embodiments, the effector construct expresses a polypeptide that modulates expression of a stress-, pathogen-, or auxin-inducible gene promoter, an epitope-tagged polypeptide, a polypeptide that represses or activates reporter gene expression, or constitutively expresses a polypeptide.

In another aspect, the invention features a plant protoplast including a reporter gene construct operably linked to a promoter of a gene of interest and (ii) an effector construct that expresses a polypeptide. In preferred embodiments, the reporter gene construct expresses a green fluorescent protein, a luciferase, or a β-glucuronidase; and the promoter of the gene of interest is inducible. In still other preferred embodiments, the protoplasts are maize or Arabidopsis protoplasts. Preferably, the protoplasts are mesophyll protoplasts. In addition, either the gene of interest or the effector construct or both encodes a signaling pathway polypeptide.

In another aspect, the invention features a high throughput assay for rapidly screening a library of nucleic acid molecules to identify a gene product that modulates expression of a gene of interest. The assay, in general, involves: (a) introducing into one or more plant protoplasts (i) a reporter gene construct operably linked to a promoter of a gene of interest and (ii) a member of a library of nucleic acid molecules, wherein the library member is expressed in the plant protoplasts; and (b) screening the protoplasts to determine whether the amount of gene expression of the reporter gene construct changes in response to the expression of the library member, a change in gene expression of the reporter gene construct identifying the gene product expressed by the library member as one that modulates expression of the gene of interest.

In preferred embodiments, the assay includes treating the protoplasts with a biotic or an abiotic stress; or treating the protoplasts with a hormone, a metabolite, or with a pathogen, elicitor, or elicitin. In other preferred embodiments, the gene of interest expresses a signaling pathway polypeptide. And in still other preferred embodiments, the reporter gene and the library member are introduced into the protoplasts by electroporation or polyethylene glycol transformation methodologies. Preferably, the protoplasts are mesophyll protoplasts, such as those prepared from the leaves of maize or Arabidopsis. In preferred embodiments, the assay includes at least approximately 10² protoplasts, or includes approximately 10⁴ to 10⁶ protoplasts.

In other preferred embodiments, the reporter gene expresses a green fluorescent protein, a luciferase, or a P-glucuronidase, and is operably linked to an inducible promoter (e.g., promoter that regulates expression of a signaling pathway). Such promoters are typically hormone-, stress-, metabolite-, light-, pathogen-, or elicitor-inducible promoters. In other embodiments, the promoter of the gene of interest is a repressible promoter.

In still other preferred embodiments, the library of nucleic acid molecules is a genomic, cDNA, expressed sequence tagged, or randomized synthetic library having at least 10² members. Preferably, at least ten different members of the library of nucleic acid molecules are introduced into the protoplasts. In addition, the methods of the invention further include determining the sequence of the member of the nucleic acid library that modulates reporter gene expression.

In yet other preferred embodiments, the library member expresses a polypeptide or an RNA molecule that modulates expression of the gene of interest; a signaling pathway polypeptide; a gene product that represses reporter gene expression; or a gene product that activates reporter gene expression.

In another aspect, the invention features a high throughput assay for rapidly screening a library of nucleic acid molecules to identify a gene product that complements a mutant phenotype. The assay, in general, involves: (a) introducing into one or more mutant plant protoplasts a member of a library of nucleic acid molecules, wherein the library member is expressed in the protoplasts; and (b) screening the mutant protoplasts to determine whether the phenotype changes in response to the expression of the library member, a phenotypic change in the protoplast identifying the gene product expressed by the library member as one that complements the mutant phenotype.

In preferred embodiments, the library member is introduced into mesophyll protoplasts (e.g., maize or Arabidopsis) by electroporation or polyethylene glycol transformation; and the assay includes at least approximately 10² protoplasts. In other preferred embodiments, the assay includes approximately 10⁴ to 10⁶ protoplasts.

Exemplary plants which are useful in the methods of the invention, as well as for generating the transgenic plants (or plant cells, plant components, plant tissues, or plant organs) of the invention, include dicots and monocots, such as sugar cane, wheat, rice, maize, sugar beet, barley, manioc, crucifer, mustard, potato, soybean, sorghum, cassava, banana, grape, oats, tomato, millet, coconut, orange, rye, cabbage, apple, eggplant, watermelon, canola, cotton, carrot, garlic, onion, pepper, strawberry, yam, papaya, peanut, onion, legume, bean, pea, mango, and sunflower.

By “polypeptide” is meant any chain of amino acids, regardless of length or post-translational modification (for example, glycosylation or phosphorylation).

By “recombinant” is meant a nucleic acid (e.g., DNA) that, is free of the genes which, in the naturally-occurring genome of the organism from which the nucleic acid of the invention is derived, flank the gene. The term therefore includes, for example, a gene or fragment thereof that is incorporated into a vector, into an autonomously replicating plasmid or virus, or into the genomic DNA of a prokaryote or eukaryote, or that exists as a separate molecule (for example, a cDNA or a genomic or cDNA fragment produced by PCR or restriction endonuclease digestion) independent of other sequences. It also includes a nucleic acid which is part of a hybrid gene encoding additional polypeptide sequence.

By “reporter gene” is meant a gene whose expression may be assayed; such genes include, without limitation, β-glucuronidase (GUS), luciferase (LUC), chloramphenicol transacetylase (CAT), green fluorescent protein (GFP), and β-galactosidase.

By “promoter” is meant any minimal sequence sufficient to direct transcription in a plant cell. Included in the invention are promoter elements that are sufficient to render promoter-dependent gene expression controllable for cell-, tissue-, or organ-specific gene expression, or elements that are inducible by external signals or agents for initiating a signaling pathway (for example, light-, pathogen-, wound-, stress-, or hormone-inducible elements or chemical inducers) or elements that are capable of cycling gene transcription or regulating gene expression of a signaling pathway; such elements may be located in the 5′ or 3′ regions of the native gene or engineered into a transgene construct.

By “operably linked” is meant that a gene and a regulatory sequence(s) are connected in such a way as to permit gene expression when the appropriate molecules (for example, transcriptional activator proteins) are bound to the regulatory sequence(s).

By “plant cell” is meant any self-propagating cell bounded by a semi-permeable membrane and containing a plastid. Such a cell also requires a cell wall if further propagation is desired. Plant cell, as used herein, includes, without limitation, algae, cyanobacteria, seeds, suspension cultures, protoplasts, embryos, meristematic regions, callus tissue, leaves, roots, shoots, gametophytes, sporophytes, pollen, and microspores.

By “signaling pathway” is meant a biological pathway mediating a cellular response to an external stimulus (e.g., an abiotic or biotic stress). Exemplary signaling pathways in plants include, without limitation, hormone (e.g., auxin, cytokinin, ABA, ethylene, jasmonic acid, or brassinosteroids), stress (e.g., cold, heat, salt, osmotic, ethanol, or H₂O₂), metabolite (e.g., glucose, sucrose, sugar analogues, acetate or other regulatory metabolites), light (e.g., red, blue, UV, and far red light), and pathogen or elicitor signaling pathways. Integral members of plant signaling pathways serve as novel targets for manipulating plant growth and development.

By “transgene” is meant any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

By “transgenic” is meant any cell which includes a nucleic acid sequence (e.g., a recombinant DNA sequence) which is inserted by artifice into a cell and becomes part of the genome of the organism which develops from that cell. As used herein, the transgenic organisms are generally transgenic plants and the DNA (transgene) is inserted by artifice into the nuclear or plastidic genome.

Other features and advantages of the invention will be apparent from the following description of the preferred embodiments thereof, and from the claims.

### DETAILED DESCRIPTION

The drawings will first be described.

### DRAWINGS

FIG. 1 shows a series of photographs demonstrating stress signaling in maize leaf protoplasts as visualized by green-fluorescent (GFP) expression.

FIG. 2 shows a series of photographs demonstrating that intracellular Ca²⁺ elevation activates stress signaling.

FIG. 3A shows a schematic illustration of the structural comparison between plant CDPKs and mammalian CaMKII.

FIG. 3B shows the sequence comparison among the kinase domains of four ATCDPKs (SEQ ID NOS:______). Identical amino acids are highlighted.

FIG. 3C shows the schematic illustrations of various PK constructs.

FIG. 3D shows a photograph of a gel illustrating the immunoprecipitation of eight PKs with anti-HA. Lane 0, background control; Lane 1, ATCDPK; Lane 2, ATCDPK1; Lane 3, ATCDPK1a; Lane 4, ATCDPK2; Lane 5, ATPKa; Lane 6, ATPKb, Lane 7, ASK1; Lane 8, ASK2, Lane M, protein molecular weight markers (92, 66, 43, 27, 18, 14 kD).

FIG. 3E shows a graph demonstrating that ATCDPK1 and ATCDPK1a activate stress-inducible transcription.

FIG. 4A shows a photograph of a gel illustrating the immunoprecipitation of ATCDPK1 and ATCDPK1 (K40M) mutant proteins.

FIG. 4B shows a series of photographs demonstrating that the ATCDPK1 (K40M) mutant does not activate stress signaling.

FIG. 4C shows a graph demonstrating that PP2C blocks the action of CDPK1.

FIG. 4D shows a schematic illustration of a model for stress signal transduction in plant cells.

FIGS. 5A-5C show the nucleotide and amino acid sequences of the ATCDPK1a PK domain, SEQ ID NO:______ and SEQ ID NO:______, respectively.

FIG. 6A is a panel of photomicrographs showing auxin responses in maize protoplasts. The protoplasts were transfected with plasmid DNA carrying either the “GH3-sGFP” or “CAB5-sGFP” auxin-response reporter construct and incubated without or with auxin. Protoplasts expressing GFP were bright green under UV light. Untransfected and uninduced protoplasts showed only blue and pink autofluorescence.

FIG. 6B is a histogram showing that the GH3 promoter and the ER7 auxin responsive element are regulated in maize protoplasts. The protoplasts were transfected with plasmid DNA carrying GH3-GUS (designated “GH3 ”), ER7-GUS (designated “ER7 ”), mutated ER7-GUS (designated “mER7”), or a GUS construct under the transcriptional control of the CaMV 35S minimal (−72) promoter (designated “35Smin”). A construct carrying the maize CAB5 promoter (Ulmasov et al., Science 276: 1865-1868, 1997) fused to the luciferase gene (designated “CAB-LUC”) was used as an internal control in each transfection. The protoplasts were incubated without or with auxin. In each treatment the GUS activity of the cell lysate was divided by the LUC activity, thereby normalizing the data for variation in experimental conditions (that is, number of cells, transformation efficiency, and cell viability). Because of differences in the basal level of expression, GUS/LUC activity of the protoplasts transfected with each construct and incubated without auxin was set to 1. The results shown were the means of triplicate samples ±SD. All experiments were repeated two to three times with similar results.

FIG. 7A is a photograph of an autoradiogram showing the expression of different protein kinases in maize protoplasts.

FIG. 7B is a photograph of an autoradiogram showing the phosphorylation activity of different protein kinases.

FIG. 7C is a photomicrograph showing that constitutively active NPK1 represses the auxin-inducible GH3 promoter. Maize protoplasts were co-transfected with the GH3-sGFP reporter and an effector construct carrying various protein kinases as indicated or vector DNA (control), and incubated with auxin to induce the GH3 promoter.

FIG. 7D is a histogram showing that constitutively active NPK1 represses auxin responsive promoters. Maize protoplasts were co-transfected with GH3-GUS (designated “GH3”) or ER7-GUS (designated “ER7”) reporter and an effector construct carrying the wild-type (designated “NPK1”) or mutated (designated “NPK1mut”) kinase domain of NPK1, or vector mutated DNA (designated “control”), and incubated with auxin. A CAB-LUC construct was used as an internal control in each transfection to normalize the GUS activity. The GUS/LUC activity of the control protoplasts induced by auxin was set to 100%. The results shown were the means of triplicate samples ±SD.

FIG. 7E is a panel showing a photograph of the expression levels of NPK protein and the null mutation of NPK1 at various times during heat shock (upper panel) and a histogram showing the activation of the GH3 promoter in the presence or absence or auxin (lower panel). The wild-type (NPK1) or mutated (NPK1mut) kinase domain of NPK1 was fused to a DHA tag (Sheen, Science 274: 1900-1902, 1996) and inserted into a plant expression vector with a heat shock inducible promoter (designated “HSP”) (Sheen et al., Plant J. 8: 777-784, 1995). Protoplasts were co-transfected with the GH3-GUS reporter and HSP-NPK1 or HSP-NPK1mut effector. CAB-LUC was used as an internal control in each co-transfection to normalize the GUS activity. The expression of the NPK1 or NPK1mut protein was induced at 40° C. for 10, 20, or 60 minutes. The protoplasts from each treatment were divided equally for protein labeling and immunoprecipitation, and for incubation without or with auxin to measure GUS/LUC activity. The GUS/LUC activity of the transfected protoplasts incubated with auxin without heat shock was set to 100%. The results shown were the means of triplicate samples ±SD. All experiments were repeated three times with similar results.

FIG. 8A is a schematic diagram showing different NPK1 constructs. The constructs carry the coding region of (1) kinase domain only, (2) NH₂-terminus and kinase domain, (3) kinase domain and COOH-terminus, and (4) full-length NPK1 protein.

FIG. 8B is a photograph of an analysis showing the levels of protein expression of the NPK1 constructs (1, 2, 3, and 4) in maize protoplasts.

FIG. 8C is a histogram showing the effect of various NPK1s on the GH3 promoter activity. Maize protoplasts were co-transfected with the GH3-GUS reporter construct and one of the NPK1 constructs (1, 2, 3 or 4) shown in FIG. 8A or vector DNA (control). CAB-LUC was used as an internal control in each transfection to normalize the GUS activity. The GUS/LUC activity of the control protoplasts in the presence of auxin was set to 100%. The results shown were the means of triplicate samples ±SD. All experiments were repeated three times with similar results.

FIG. 9A is a panel showing the results of a MAPK in-gel assay (upper panel) and a histogram showing kinase activity (lower panel) of maize protoplasts expressing different MAPKKKs. Protoplasts were transfected with (1) vector DNA for background control; (2) NPK1 kinase domain construct; (3) NPK1 kinase domain mutant construct; (4) full-length NPK1 construct; and (5) CTR1 kinase domain construct. The radioactivity of the 44 kDa putative MAPK band was quantified using a Phosphorimager and normalized to the signal from the background control.

FIG. 9B is a photograph of a gel electrophoretic analysis showing the activity of anti-MAPK immunoprecipitated proteins. Protoplasts were transfected with (1) vector DNA for background control; (2) NPK1 kinase domain construct; and (3) NPK1 kinase domain mutant construct.

FIG. 9C is a panel of gel electrophoretic analyses showing that MAPK phosphatase (MKP1) inactivates NPK1-induced MAPK. Protoplasts were co-transfected with NPK1 and various protein phosphatase (PP) constructs. The transfected protoplasts were divided to determine protein expression level (upper panel), and to perform the kinase in-gel assay (lower panel).

FIG. 9D is a panel of photomicrographs of maize protoplasts showing that MKP1 abolishes the NPK1 repression of the auxin-inducible transcription. Protoplasts were co-transfected with the GH3-sGFP reporter and NPK1, NPK1+MKP1, NPK1+PP1, NPK1+PP2A, or NPK1+PP2C, and incubated in a medium with auxin. All experiments were repeated two to three times with similar results.

FIG. 10A is a histogram showing the H₂O₂, heat shock, and ABA responses in designated Arabidopsis protoplasts. Protoplasts were transfected with GST6-LUC (designated “GST6”), HSP 18.2-LUC (designated “HSP 18.2”), or RD29A-LUC (designated “RD29A”) reporter constructs. The transfected protoplasts were divided (105 per sample) and incubated at 23° C. without (−) or with (+) 200 μM of H₂O₂, 38° C. (heat), or 100 μM ABA for 3 hours. The CaMV35S-GUS reporter construct was used as an internal control in each transfection to normalize data for differences in transfection efficiency and cell viability. LUC/GUS was measured as an indicator of the promoter activities. The induction of the HSP 18.2 promoter was about 1000-fold, due to extremely low basal expression level. Data are the results of triplicate samples and three independent experiments.

FIG. 10B is a histogram showing that H₂O₂ activated two oxidative stress-induced promoters. Arabidopsis protoplasts were transfected with GST6-LUC (GST6), HSP18.2-LUC (HSP18.2), RD29A-LUC (RD29A) or CaMV35S-LUC (35S) reporter constructs, and incubated without (−) or with (+) 200 μM of H₂O₂ for three hours before the promoter activities were measured. Data are the results of triplicate samples and three independent experiments.

FIG. 10C shows that H₂O₂ induces two putative MAPKs. Arabidopsis protoplasts were treated with 200 μM H₂O₂ or water for 10, 15, and 30 minutes. The MAPK in-gel kinase activity assay.

FIG. 10D is a histogram showing that H₂O₂ and heat shock suppress the auxin responsive GH3 promoter. Arabidopsis protoplasts were transfected with the GH3-GUS reporter construct, divided (10⁵ protoplasts per sample), and incubated in the absence (− auxin) or presence of 1 μM NAA (+ auxin) and 200 μM of H₂O₂, or 100 μM ABA at room temperature or at 38° C. (heat) for 3 hours. A CaMV35S-LUC reporter construct was used as an internal control. GUS/LUC was measured as an indicator of GH3 promoter activity. Data are the results of triplicate samples and three independent experiments. Similar results were obtained when GH3-LUC reporter was used.

FIG. 11A is a photograph of an autoradiogram showing the expression of the ANP kinases. Arabidopsis protoplasts were transfected with an effector construct expressing one of the HA-tagged protein kinases: kinase domain of ANP1 (designated “ΔANP1”), kinase domain of ANP2 (designated “ΔANP2”), kinase domain of ANP3 (designated “ΔANP3”), kinase domain of ANP1 mutated in the ATP binding site (designated “ΔANP1 m”), and full-length ANP1 (ANP1). The transfected protoplasts were incubated in the presence of [35S]-methionine for 4 hours to allow expression and labeling of the effector proteins. The HA-tagged kinases were immunoprecipitated, separated by SDS-PAGE, and detected.

FIG. 11B is a photograph of an autoradiogram showing that ANPs activate two endogenous MAPKs in Arabidopsis. Arabidopsis protoplasts were transfected with the ANP constructs described in FIG. 6A or with a vector (control) and incubated for 4 hours to allow expression. Activity of endogenous MAPKs in the transfected cells was detected by an in-gel assay using myelin basic protein (MBP) as a substrate.

FIG. 11C is a photograph of an autoradiogram showing that ANP1 induced AtMPK3 and AtMPK6 in vivo. Arabidopsis protoplasts were transfected with constructs expressing one of the HA-tagged Arabidopsis MAPKs (designated “AtMPK2 to 7”) alone, or co-transfected with another construct expressing HA-tagged ANP1 kinase domain (designated “ΔANP1”). The transfected cells were divided (10⁵ each) to detect protein levels (upper panel) or to assay the MAPK activity after immunoprecipitation by using MBP as a substrate (lower panel). Stars indicate non-specific bands seen after immunoprecipitation.

FIG. 11D is a photograph of an autoradiogram showing that stresses activate AtMPK3 and ANP1. Arabidopsis protoplasts were transfected with AtMPK3 construct alone or co-transfected with full-length ANP1 (designated “AtMPK3+ANP1”) or active ANP1 (designated “AtMPK3+ΔANP1”). Cells were incubated for 4 hours to allow protein expression. The protoplasts (10⁵ each) were treated with 200 μM of H₂O₂, 38° C. (designated “heat”), 4° C. (designated “cold”), 1 μM NAA (designated “auxin”), or 100 μM ABA for 15 minutes. The AtMPK3 was immunoprecipitated using an anti-HA antibody and assayed for activity by using MBP as a substrate. All data presented in the figure were repeated at least three times with similar results.

FIG. 12A is a histogram showing the response of different dicot promoters to the constitutive expression of the ANP1 kinase domain in Arabidopsis protoplasts. Protoplasts were co-transfected with either the NR2-LUC (designated “NR2”), AS1-LUC (designated “AS1”), RD29A-LUC (designated “RD29A), HSP-LUC (designated “HSP”), CAB2-LUC (designated “Cab2”), CHSP-LUC (designated “CHSP”), or GST6-LUC (designated “GST6”) reporter gene constructs and an effector construct carrying the wild-type (kANP1) kinase domain, mutated (NPK1mut) kinase domain of NPK1, or the kinase domain of CTR1 (designated “kCTR1”). A 35S NPKmut-GUS construct was used as an internal control in each transfection to normalize the LUC activity. The LUC/GUS activity of the NPK1mut was set to 1. The results shown were the means of triplicate samples ±SD.

FIG. 12B is a histogram showing that ANP1 activates stress-inducible promoters. Arabidopsis protoplasts were co-transfected with one of the reporter constructs: GST6-LUC (designated “GST6”), HSP 18.2-LUC (designated “HSP18.2”), or RD29A-LUC (designated “RD29A”) and one of the effector constructs as described in FIG. 6A, kinase domain of CTR1 (desingated “ΔCTR1”), kinase domain of ASK1 (designated “ΔASK1”), full-length CK1-1 (designated “CK1-1”), or a vector (“control”). The CaMV35S-GUS reporter construct was used as an internal control. Transfected cells were incubated for 6 hours before LUC/GUS was measured as an indicator of the promoter activity. Data are the results of triplicate samples and three independent experiments.

FIG. 12C is a histogram showing that ANPs repress the auxin response. Arabidopsis protoplasts were co-transfected with the GH3-GUS reporter construct and one of the effector constructs as described in FIG. 6A, kinase domain of CTR1 (designated “ΔCTR1”), kinase domain of ASK1 (designated “ΔASK1”), full-length CK1-1 (designated “CK1-1”), or a vector (designated “control”). The CaMV35S-LUC reporter construct was used as an internal control. The transfected protoplasts were incubated for 3 hours to allow effector expression before the induction by 1 μM NAA for 3 hours. GUS/LUC was measured as an indicator of the GH3 promoter activity. Data are the results of triplicate samples and three independent experiments.

FIG. 13A is a histogram showing the seed germination frequencies of different transgenic lines of tobacco expressing NPK1. Wild-type (wt) and three independent transgenic lines (2A, 3B, 4A) were examined. The results shown are the means of triplicate samples, 100 seeds each, ±SD.

FIG. 13B is a panel of photomicrographs showing the morphological analysis of wild-type and line 4A transgenic seeds. The wild-type (upper panel, labeled 1, 2, 3, and 4) and 4A (lower panel, labeled 5, 6, 7, and 8) seeds were soaked for 24 hours in water. The seeds are shown as a population (1,5), typical single seed (2,6), dissected (3,7), and used for the embryo isolation (4,8). The wild-type (3), but not the transgenic (7) seeds, showed abundant endosperm, noticeable after the dissection. At least 10 seeds from each population were analyzed in this study.

FIG. 13C is a photograph of an RNA blot analysis of the NPK1 transgene expression in wild-type and transgenic tobacco. RNA was isolated from two week-old seedlings. The NPK1 probe hybridized with the transgene RNA only. The endogenous NPK1 mRNA was not detected. Ubiqutin (designated “UBQ”) expression was used as a control.

FIG. 13D is a photograph of a protein blot analysis of transgene expression. The same amount of proteins (50 mg per lane), extracted from two week-old seedlings, were fractionated in the 12% SDS-PAGE gel and blotted. HA antibody was used to detect HA-tagged transgene proteins. A tobacco transgenic line overexpressing a HA-tagged MEK protein (MEK) was used as a positive control.

FIG. 14 is a photograph showing the drought resistance of transgenic tobacco plants expressing the NPK1 transgene. Wild-type tobacco seedlings are shown on the left; seedlings of transgenic tobacco, line NPK1-A4, are shown on the right.

FIG. 15A is a photograph showing the stress tolerance of transgenic tobacco plants expressing NPK1. Wild-type (WT) and transgenic (2A, 3B, 4A) plants were germinated and grown on a ¼ MS medium for 3 weeks.

FIG. 15B is a photograph showing the tolerance of transgenic tobacco plants expressing NPK1 to freezing temperature. Wild-type (WT) and the transgenic (2A, 3B, 4A) plants were grown on plates for 10 days before freezing termperature treatment (−10° C., 3 hours). The photograph was taken 11 days after treatment.

FIG. 15C is a photograph showing salt stress tolerance of transgenic tobacco plants expressing NPK1. Wild-type (WT) and transgenic plants (2A, 3B, 4A) were germinated on ¼ MS medium for 6 days, and then transferred to plates containing 300 mM of NaCl for 3 days. The photograph was taken 11 days after the plants were transferred back to the MS plates without NaCl. The graph represents data from five plates (each plate had 10 plants of each genotype).

FIG. 15D is a photograph showing the tolerance of transgenic tobacco plants expressing NPK1 to heat shock. Wild-type (WT) and transgenic (2A, 3B, 4A) plants were grown on plates for 10 days before heat treatment (48° C., 45 minutes). The photograph was taken 18 days after treatment. The graph represents the data from five plates (each plate had 10 plants of each genotype).

### I. STRESS RESPONSES IN PROTOPLASTS

Evidence is presented below showing that constitutively active mutants of two closely related Ca²⁺ dependent protein kinases (CDPK1 and CDPK1a) activate a stress-inducible promoter, bypassing stress signals. The effects of CDPK1 and CDPK1a are specific since six distinct plant protein kinases, including two other CDPKs, failed to mimic stress signaling. The activation is abolished by a CDPK1 mutation in the kinase domain, and diminished by a constitutively active protein phosphatase 2C capable of blocking the stress hormone ABA responses. The results indicate that CDPKs (including their PK domains) play distinct physiological roles. CDPK1 and CDPK1a are therefore examples of positive regulators for controlling stress signal transduction in plants. Expression of such regulators in transgenic plants is useful for turning on the stress signal transduction pathway as a means for increasing plant tolerance to multiple stress conditions, including drought, salinity, and extreme temperature conditions.

Stress Signaling in Maize Leaf Protoplasts Visualized by GFP Expression

Responses to multiple stress treatments were monitored using green-fluorescent protein (GFP) as a vital reporter (Chiu et al. Curr. Biol. 6:225, 1996), using a single cell maize leaf protoplast system (Sheen, EMBO J. 12:3497, 1993). A chimeric gene was generated by fusing the stress-inducible CDPK HVA1 promoter (Straub et al., Plant Mol. Biol. 26:617, 1994) to a synthetic GFP sequence (HVA1-SGFP) (Chiu et al., Curr. Biol. 6:225, 1996). The barley HVA1 promoter was obtained by PCR using barley genomic DNA and two primers: 5′TCCACCGAGATGCCGACGCA-3′ (SEQ ID NO:) and 5′-GTTGGAGGCCATGGTCGTCTCACGAT-3′ (SEQ ID NO:). The HVA1 promoter and the SGFP were fused at the ATG NcoI site. The CDPK HVA1 gene has been reported to be activated by multiple stress signals in vegetative tissues (Straub et al., Plant Mol. Biol. 26:617, 1994). Four clones were selected and tested for stress responses with identical results as is discussed below.

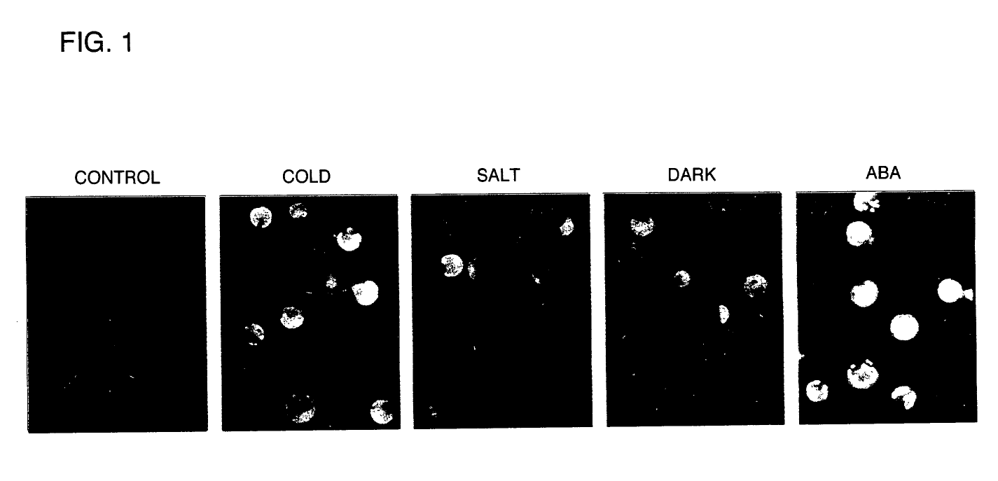

Maize leaf protoplasts were electroporated with the plasmid DNA carrying HVA1-SGFP and divided (10⁵ cells/ml per sample) for various treatments: constant light (15 μm⁻²S⁻¹) at 23° C. for sixteen hours (Control), 0° C. for four hours followed by twelve hours at 23° C. (Cold), 0.2 M NaCl for three hours, washed, and incubated for thirteen hours (Salt), constant darkness for sixteen hours (Dark), and 100 μM ABA for sixteen hours (ABA) (FIG. 1). The protocol for transient expression analysis using maize leaf protoplasts has been described by Sheen (EMBO J. 12: 3497, 1993) and Chiu et al. (Curr. Biol. 6:225, 1996). About 10⁵ protoplasts from each treatment were observed using a fluorescence microscope as described by Chiu et al. (Curr. Biol. 6:225, 1996). The experiment was repeated three times with similar results. About 50% of the protoplasts, showing green/yellow fluorescence after the induction, were transiently transformed. Control and untransfected protoplasts showed red autofluorescence from chlorophyll. GFP expression was visible with 1 μM ABA (data not shown).

In addition, after electroporation of the plasmid DNA carrying HVA1-SGFP into maize leaf protoplasts, the expression of GFP was found to be enhanced by cold, high salt, dark, and ABA (FIG. 1). These responses were specific to HVA1-SGFP because the expression of an internal control, generated by fusing the maize ubiquitin promoter (Christensen et al., Plant Mol. Biol. 18:675, 1992) and the β-glucuronidase gene (UBI-GUS) (Jefferson, Plant Mol. Biol. Rep. 5:387, 1987), was not affected (data not shown). In addition, the GFP expression derived from UBI-SGFP was not changed by the same treatments (data not shown).

Intracellular Ca²⁺ Elevation Activates Stress Signaling

The role of Ca²⁺ as a second messenger in multiple stress responses was also studied. The effects of increased intracellular Ca²⁺ on HVA1-SGFP expression in maize leaf protoplasts using Ca²⁺ ionophore was examined using standard methods as described below (Knight et al., Nature 352:524, 1991; Schroeder and Thuleau, Plant Cell 3:555, 1991; Braam, Proc. Natl. Acad. Sci. U.S.A. 89:3213, 1992; McAinsh et al., Plant Cell 4:1113, 1992; Assmann, Ann. Rev. Cell Biol. 9:345, 1993; Poovaiah and Reddy, Crit. Rev. Plant Sci. 12:185, 1993; Trewavas and Knight, Plant Mol. Biol. 26:1329, 1994; Ward and Schroeder, Plant Cell 6:669, 1994; Bethke et al., in Plant Hormones, Davies, Ed., (Kluwer Academic 1995) pp. 298-317; Bush, Ann. Rev. Plant Physiol. Plant Mol. Biol. 46:95, 1995; Monroy and Dhindsa, Plant Cell 7:32, 1995; and Knight et al., Plant Cell 8:489, 1996).

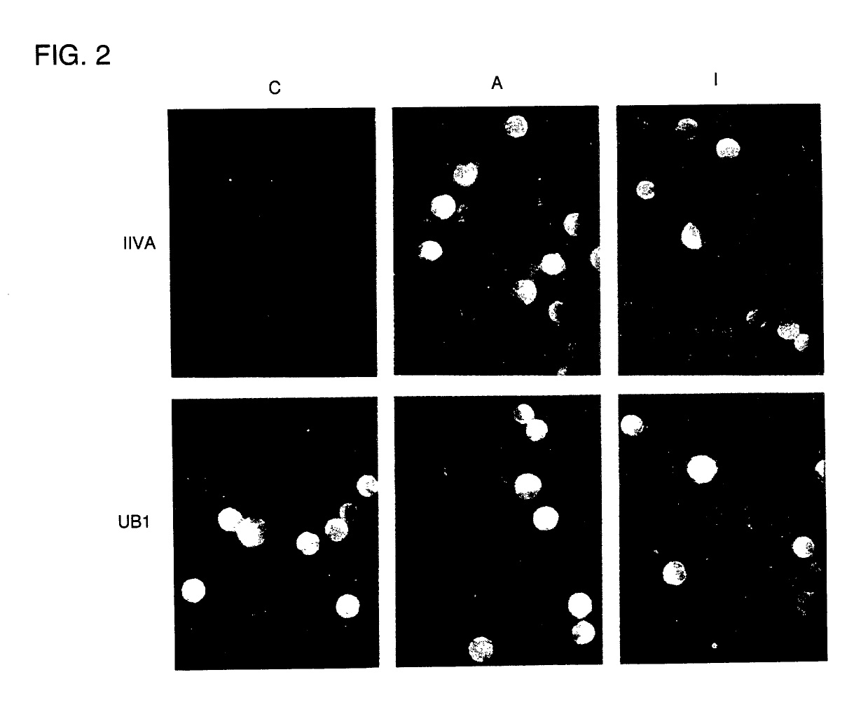

Maize leaf protoplasts transfected with HVA1-SGFP (HVA) or UBI-SGFP (UBI) were treated with 1 mM Ca²⁺, 1 mM Ca²⁺/100 nM A23187, and 1 mM Ca²⁺/100 nM ionomycin, respectively designated “C,” “A,” and “I” in FIG. 2. UBI-SGFP was constructed by inserting the UBI promoter from pAHC27 (Christensen et al., Plant Mol. Biol. 18:675, 1992) into the SGFP vector (Chiu et al., Curr. Biol. 6:225, 1996). Protoplast transient expression is the same as described by Sheen (EMBO J. 12: 3497, 1993) and Chiu et al. (Curr. Biol. 6:225,1996). About 10⁵ protoplasts from each treatment were observed using a fluorescence microscope (Chiu et al., Curr. Biol. 6:225, 1996). The experiment was repeated twice with similar results.

As shown in FIG. 2, the expression of HVA1-GFP was significantly increased by the Ca²⁺/ionomycin and Ca²⁺/A23187, but not by Ca²⁺ alone in the incubation medium. This activation is specific because the same treatment did not influence UBI-SGFP expression (FIG. 2), but was found to inhibit the expression of GFP controlled by a stress-repressible photosynthetic gene promoter (Sheen, EMBO J. 12:3497, 1993; data not shown).

Constitutive ATCDPK1 and ATCDPK1a Activate Stress Signal Transduction

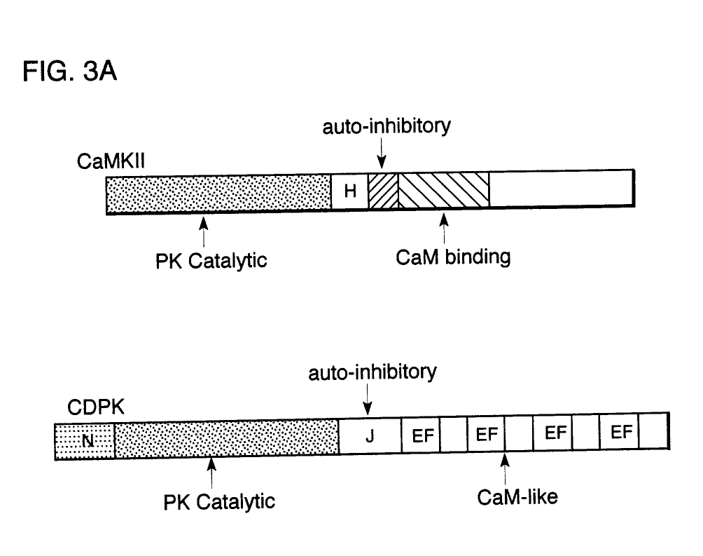

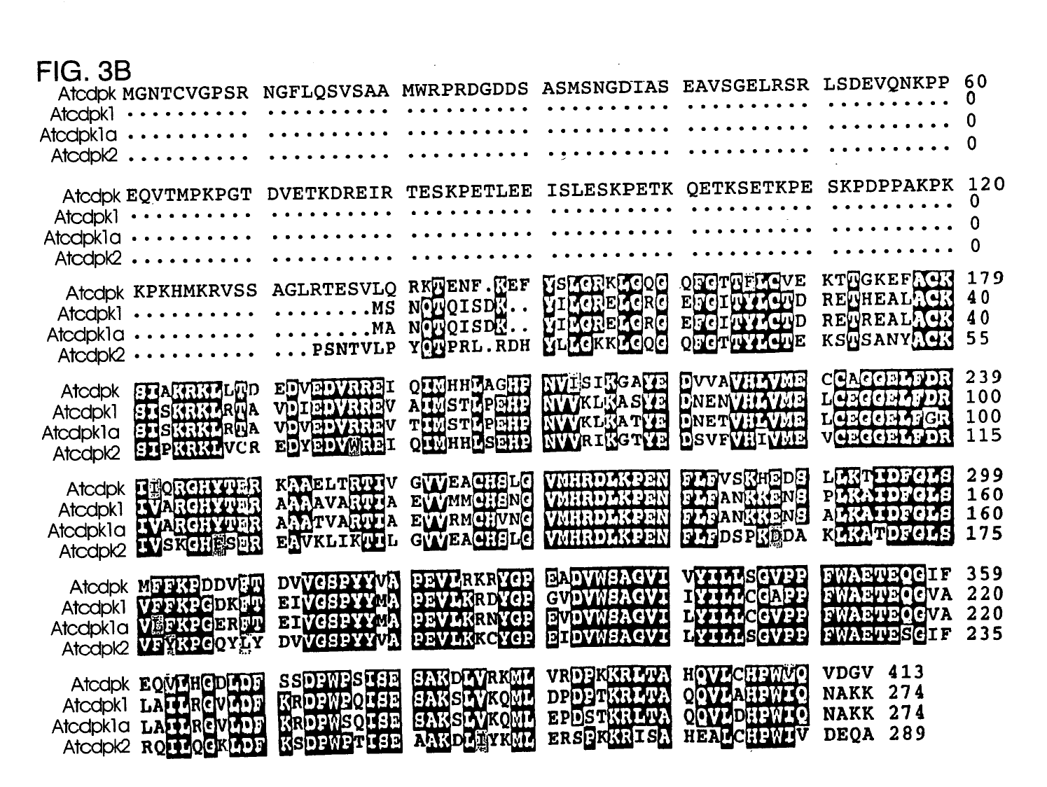

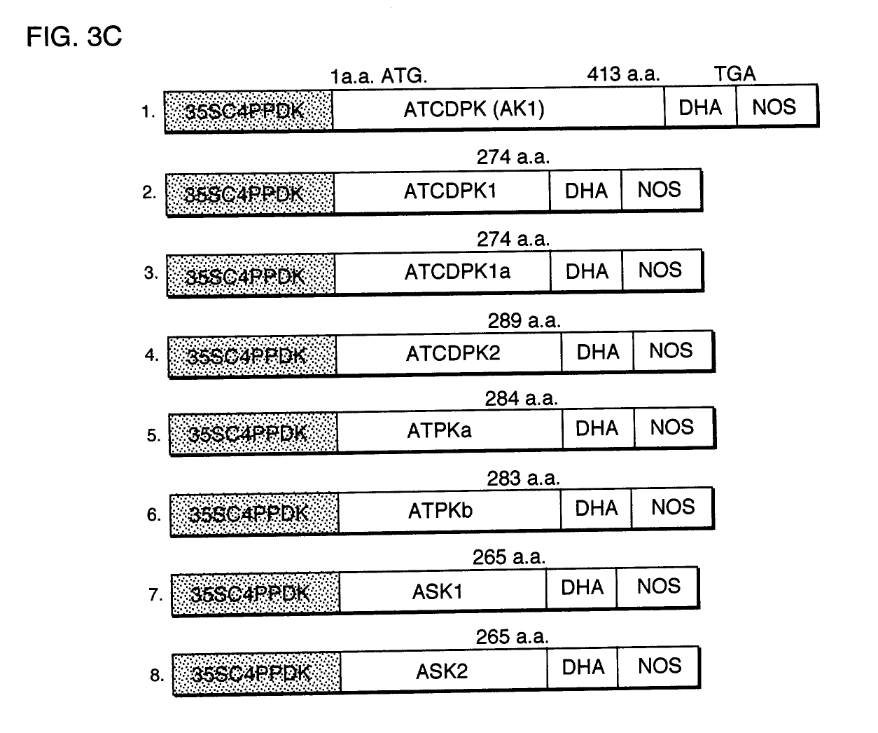

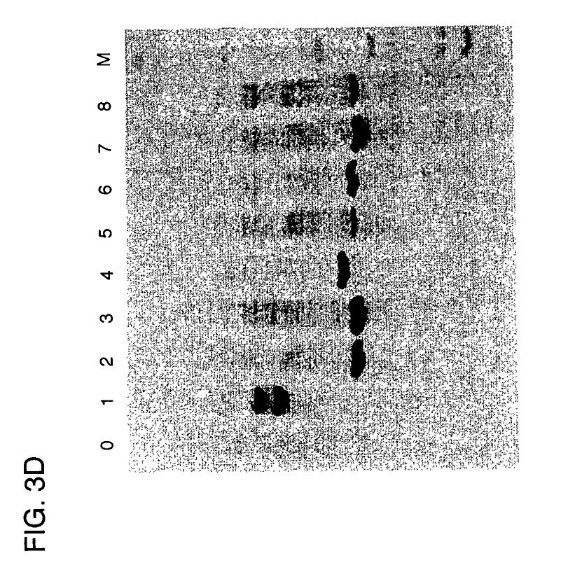

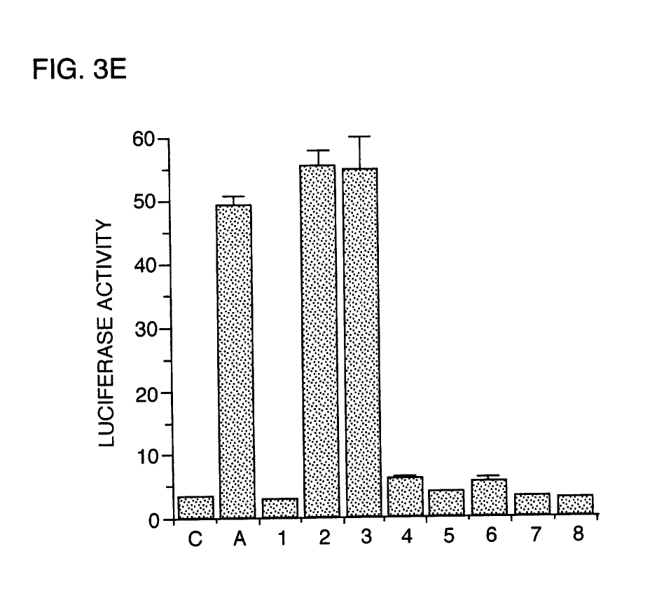

To determine whether Ca²⁺ activated protein kinases (PKs) play a role in stress signal transduction in plants, the effect of co-expressing four constitutively active Ca²⁺-dependent protein kinases (CDPKs) on the HVA1 promoter activity was examined. As is shown in FIG. 3A, plant CDPKs share extensive sequence identity with the mammalian multifunctional Ca²⁺/calmodulin-dependent KPII (CaMKII) (Kapiloff et al., Proc. Natl. Acad. Sci. USA 88:3710, 1991). However, instead of bearing a calmodulin binding site, this family of PKs carries a calmodulin-like domain at the C-terminus (FIG. 3A). This unique feature presumably allows CDPKs to respond to Ca²⁺ signals directly without calmodulin. Various other domains found in CaMKII and CDPK are shown in FIG. 3A, these include: (H) hinge, (N) NH₂ terminal domain for some CDPKs (J) junction, (EF) EF-hand Ca²⁺-binding site (Harper et al., Science 252:951, 1991; Suen and Choi, Plant Mol. Biol. 17:581, 1991; Roberts and Harmon, Ann. Rev. Plant Physiol. Plant Mol. Biol. 43:375, 1992; Estruch et al., Proc. Natl. Acad. Sci. USA 91:8837, 1994; Urao et al., Plant Physiol. 105:1461, 1994; Urao et al., Mol. Gen. Genet. 244:331, 1994; Harper et al., Bioch. 33:7267, 1994; and Kapiloff et al., Proc. Natl. Acad. Sci. USA 88:3710, 1991). Currently, CDPKs are the most prevalent serine/threonine PKs found in higher plants as the cloning of numerous CDPKs in a broad range of plant species has been reported (Harper et al., Science 252:951, 1991; Suen and Choi, Plant Mol. Biol. 17:581, 1991; Roberts and Harmon, Ann. Rev. Plant Physiol. Plant Mol. Biol. 43:375, 1992; Estruch et al., Proc. Natl. Acad. Sci. USA 91:8837, 1994; Urao et al., Plant Physiol. 105:1461, 1994; Urao et al., Mol. Gen. Genet. 244:331, 1994; Harper et al., Biochem. 33:7267, 1994).

Eight Arabidopsis PKs with full-length coding sequences were chosen for the following experiments (Urao et al., Mol. Gen. Genet. 244:331, 1994; Harper et al., Bioch. 33:7267, 1994; Minet et al., Plant J. 2:417, 1992; Anderberg and Walker-Simmons, Proc. Natl. Acad. Sci. USA 89:10183, 1992; Park et al., Plant Mol. Biol. 22:615, 1993; Holappa and Walker-Simmons, Plant Physiol. 108:1203, 1995). 1

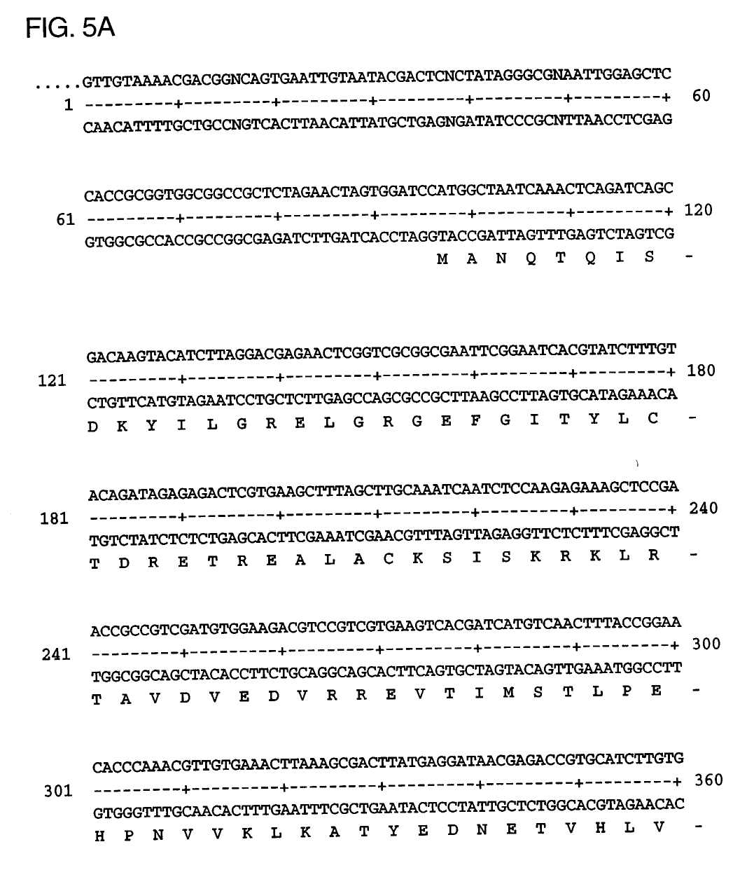

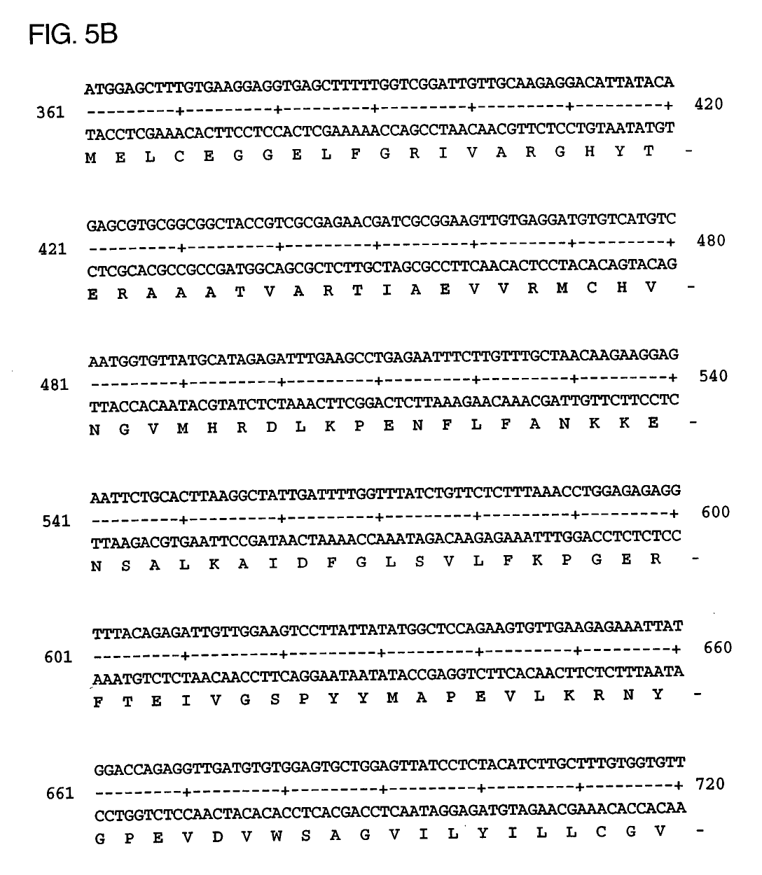

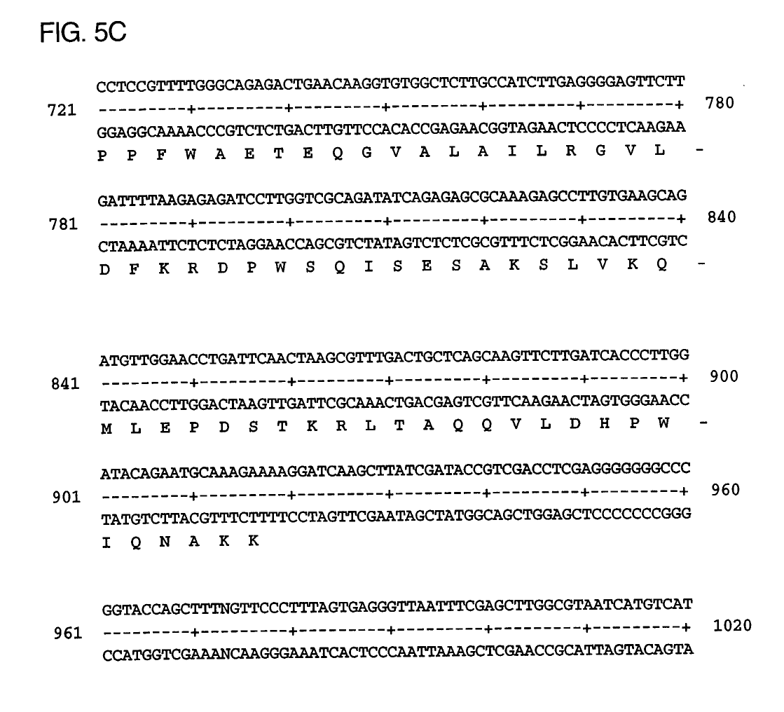

Two CDPKs (ATCDPK1 and ATCDPK1a) are closely related (96% amino acid and similarity) while the other two CDPKs (AK1/ATCDPK and ATCDPK2 have more divergent sequences (78% and 75% amino acid similarity respectively, to ATCDPK1) (FIG. 3B). In vitro assays showed that AK1/ATCDPK and ATCDPK2 possess calcium-dependent PK activity and the truncated AK1/ATCDPK has calcium-independent (constitutively active) PK activity by (Urao et al., Mol. Gen. Genet. 244:331, 1994; and Harper et al., Bioch. 33:7267, 1994). The PK activity of ATCDPK1, however, has not been demonstrated in vitro because it does not phosphorylate common PK substrates (Urao et al., Mol. Gen. Genet. 244:331, 1994). The ATCDPK1a cDNA that has restriction enzyme digestion patterns distinct from those of ATCDPK1, was identified during the isolation of ATCDPK1 by polymerase chain reaction (PCR) (Minet et al., Plant J. 2:417,1992). The nucleotide and amino acid sequences of the ATCDPK1a PK are shown in FIGS. 5A-5C (SEQ ID NO:______ and SEQ ID NO:______, respectively). The effect of four other Arabidopsis PKs (ATTPKa, ATPKb, ASKQ, ASK2) that share significant homology with the ABA-inducible PK (PKABA1), speculated to mediate ABA signal transduction, were also tested (Anderberg and Walker-Simmons, Proc. Natl. Acad. Sci. USA 89:10183, 1992; Park et al., Plant Mol. Biol. 22:615, 1993; Holappa and Walker-Simmons, Plant Physiol. 108:1203, 1995). These PK cDNAs were obtained by PCR, and at least two clones of each cDNA were used for transient expression analysis. PK cDNAs were obtained by PCR using an Arabidopsis cDNA library. The primers for these reactions were: (AK1/ATCDPK) 5′-GAAGATCTATGGGTAATACTTGTGTTGGA-3′ (SEQ ID NO:) and 5′-GTCAAGGCCTGTCGACTTGAACCCATGG-3′ (SEQ ID NO:); (ATCDPK1) and (ATCDPKA1a) 5′-GCGGATCCATGGCTAATCAAACTCAGATCAGCG-3′ (SEQ ID NO:) and 5′-GTCAAGGCCTCATCAGTGAGAACATGTTC-3′ (SEQ ID NO:); (ATCDPK2) 5′-GCGGATCCATGGAGACGAAGCCAAACCCTA-3′ (SEQ ID NO:) and 5′-GTCAAGGCCTTGCTTGTTCATCGACAATCC-3′ (SEQ ID NO:); (ATPKa) 5′-CATGCCATGGCTCCGGCGACTAATTCACCG-3′ (SEQ ID NO:) and 5′-GTCAAGGCCTATTCTTCAAGAACCATTTATCG-3′ (SEQ ID NO:); (ATPKb) 5′GCGGATCCATGGCTCGAGCTCCGGTGACCA-3′ (SEQ ID NO:) and 5′-GTCAAGGCCTATTCTTCAAGAACCAACTATG-3′ (SEQ ID NO:); (ASK1) 5′-GCGGATCCATGGCTAAGTCAGAGCTGGTGAAAG-3′ (SEQ ID NO:) and 5′-GTCAAGGCCTATTCTTTAGGAACCATGAATG-3′ (SEQ ID NO:); (ASK2 5′-GCGGATCCATGGCTAAGTATGACGTTGTCAAGG-3′ (SEQ ID NO:) and GTCAAGGCCTATTCTTCAAGTACCACGG-3′ (SEQ ID NO:). The sequence of ATCDPKA1 a was determined for both strands using an automatic sequencing facility.

Truncated forms containing all eleven PK domains, analogous to the construction of a constitutively active mutant of CaMKII in mammals (Kapiloff et al., Proc. Natl. Acad. Sci. USA 88, 7267, 1994) (FIG. 3A and FIG. 3B), were inserted into the plant expression vector with a strong constitutive promoter 35SC4PPDK (Sheen, EMBO J. 12:3497, 1993; Chiu et al., Curr. Biol. 6:225,1996). The putative regulatory domains of theses PKs were deleted (FIG. 3C). To allow convenient monitoring of protein expression, these PKs were fused in frame to a double hemagglutinin (HA) epitope tag (designated DHA in FIG. 3C) at the C-terminus and inserted into a plant expression vector (Sheen, EMBO J. 12:3497, 1993; and Chiu et al., Curr. Biol. 6:225, 1996).

The expression of eight PKs in transfected maize leaf protoplasts were demonstrated by immunoprecipitation of [³⁵S] methionine labeled proteins with the anti-HA monoclonal antibody (FIG. 3D). Transfected protoplasts were incubated for four hours to allow mRNA accumulation and then labeled with 200 μCi/ml of [³⁵S] methionine for twelve hours before harvest. Immunoprecipitation was carried out based on a published protocol by Kapiloff et al. (Proc. Natl. Acad. Sci. USA 88:3710, 1991). The proteins were separated on a 12.5% SDS-PAGE gel and visualized by fluorography. All PK constructs yielded strong bands, approximately 30-35 kD for 2-8 and around 55 kD for AK1/ATCDPK, indicating that all transgenes were expressed efficiently (FIG. 3D). For quantitating the effect of various constitutive PKs on stress signaling, another chimeric gene with the HVA 1 promoter and the luciferase coding sequence (Leuhrsen et al., Meth. Enz. 216:397, 1992) (HVA1-LUC) was generated. Co-expression experiments were performed by electroporating the reporter (HVA1-LUC) and the effector (35SC4PSK-PK-HA) plasmids together into maize leaf protoplasts as follows.

Maize leaf protoplasts were transfected with HVA1-LUC alone and incubated without (FIG. 3E, “C”) or with 100 μM ABA (FIG. 3E, “A”). HVA1-LUC was also co-electroporated with the PK constructs (1-8) shown in FIG. 3C and FIG. 3D, and incubated without ABA (FIG. 3E, “1-9”). Relative LUC activities from duplicated samples are shown. About 2% of the cell lysates were used for LUC (Luehrsen et al., Meth. Enz. 216:397, 1992) and GUS assays (Sheen, EMBO J. 12:3497, 1993; and Jefferson, Plant Mol. Biol. Rep. 5:387, 1987). The experiment was repeated three times with similar results.

The results showed that ATCDPK1 and ATCDPK1 a, but not the other six PKs, could specifically activate LUC expression controlled by the HVA1 promoter (FIG. 3E). An identical set of PKs without the HA tag gave the same results (data not shown). The expression of UBI-GUS as an internal control was not affected (data not shown).

CDPK1 Activates but PP2C Abolishes Stress Signaling

To show that PK activity is important for the activation of the stress-inducible HVA1 promoter, a null mutation was made by site-directed mutagenesis to eliminate the ATP binding site (K40) in ATCDPK1 (Urao et al., Mol. Gen. Genet. 244:331, 1994; and Kapiloff et al., Proc. Natl. Acad. Sci. USA 88:3710, 1991) and analyzed as follows. Maize leaf protoplasts were electroporated with HVA1-SGFP alone, or with ATCDPK1 (CDPK1) and the ATCDPK1 (K40M) mutant (CDPK1mut). About 10⁵ protoplasts from each treatment were observed using a fluorescence microscope (Chiu et al., Curr. Biol. 6:225, 1996). About 50% of the protoplasts were transiently transformed.

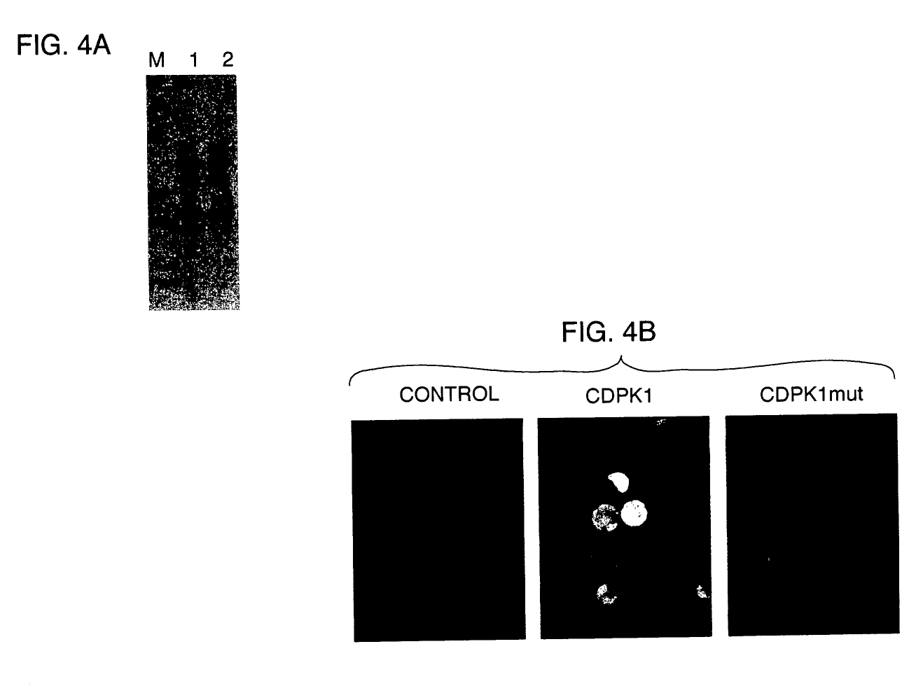

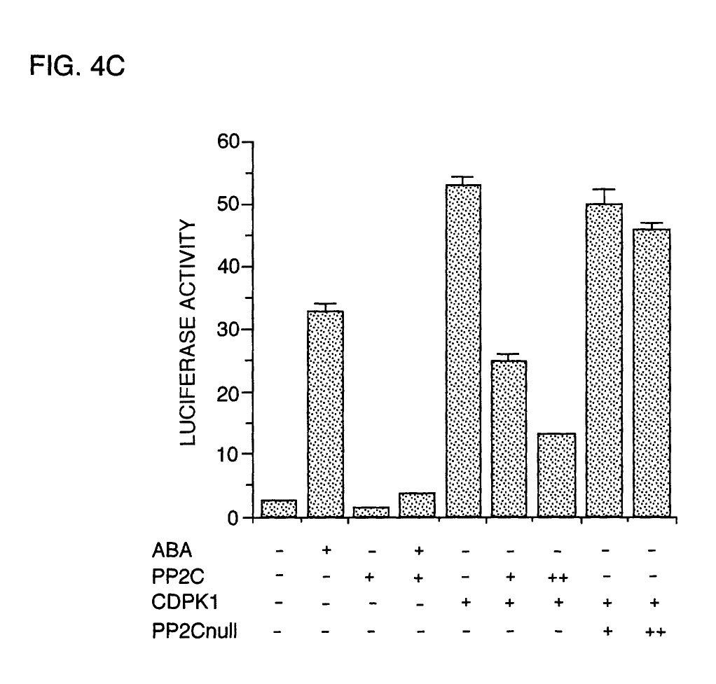

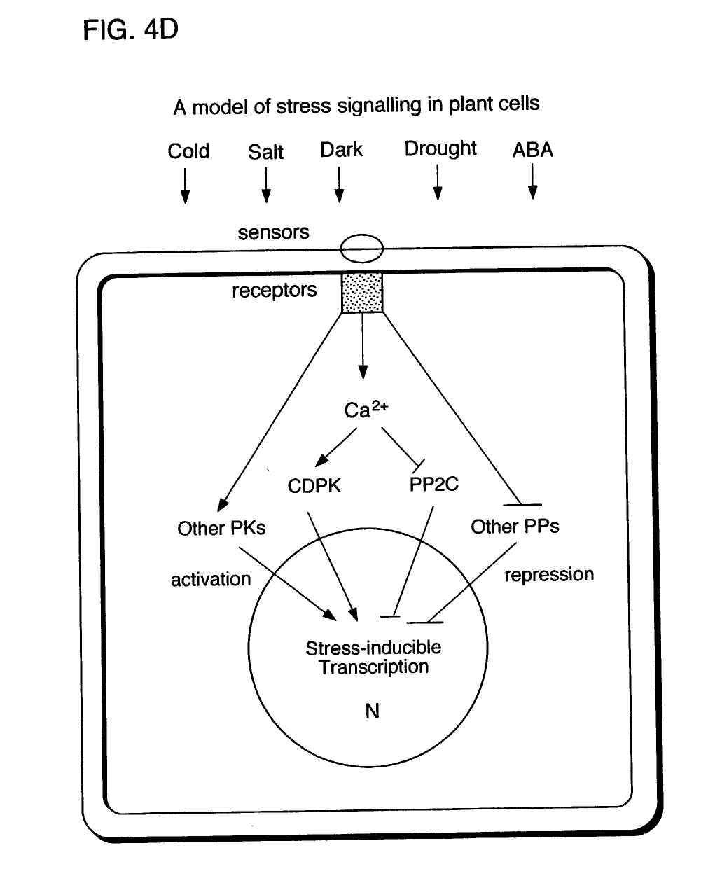

The kinase mutation (K40M) did not affect the expression of the protein (FIG. 4A), but it could no longer activate the expression of HVA1-GFP (FIG. 4B). The expression of UBI-SGFP was not affected by ATCDPK1 or the ATCDPK1 mutant (data not shown). This result indicates that the PK domain of ATCDPK1 was required and sufficient to recognize specific protein substances mediating stress signal transduction. The deleted regulatory domain was likely involved in PK activity control in response to stress signals (Harper et al., Science 252:951, 1991; Suen and Choi, Plant Mol. Biol. 17:581, 1991; Roberts and Harmon, Ann. Rev. Plant Physiol. Plant Mol. Biol. 43:375, 1992; Estruch et al., Proc. Natl. Acad. Sci. USA 91:8837, 1994; Urao et al., Plant Physiol. 105:1461, 1994; Urao et al., Mol. Gen. Genet. 244:331, 1994; Harper et al., Bioch. 33:7267, 1994; and Kapiloff et al., Proc. Natl. Acad. Sci. USA 88:3710, 1991).

To further support the idea that ATCDPK1 and ATCDPK1a are positive regulators in plant stress signal transduction, the effect of a specific and constitutively active Arabidopsis protein phosphatase 2C (PP2C) capable of abolishing ABA responses (Leung et al., Science 264:1448, 1994; Meyer et al., Science 264:1452, 1994; Armstrong et al., Proc. Natl. Acad. Sci. USA 92:9520, 1995) was examined as follows. Maize leaf protoplasts were electroporated with HVA1-LUC alone or with the effectors as indicated. PP2C null did not show PP2C activity (data not shown). The experiments with the constitutively active PP2C or the null PP2C were performed with two concentrations of plasmid DNA (+, 20 μg and ++, 60 μg) (FIG. 4C). LUC and GUS assays were performed with 2% of the cell lysates as described previously by Sheen (EMBO J. 12:3497, 1993), Jefferson (Plant Mol. Biol. Rep. 5:387, 1987), and Luehrsen et al. (Meth. Enz. 216:397, 1992). Relative LUC activities from duplicated samples are shown. The experiment was repeated three times with similar results (FIG. 4C).

HVA1-LUC expression activated by ABA was significantly repressed by the constitutively active PP2C. Constitutive PP2C, but not its null version diminished, but did not completely abolish, HVA1-LUC expression enhanced by ATCDPK1 (FIG. 4C). Other serine/threonine PPs such as PP1, PP2A and PP2B might be required to completely counteract the effect of ATCDPK 1, which could be a convergent point of multiple stress signaling (FIG. 4D). The same results were obtained with ATCDPK1A, and the expression of the internal control UBI-GUS were not affected (data not shown). As the genes involved in stress responses are highly conserved in plants, the role of ATCDPK1 and ATCDPK1a in stress signal transduction may extend to various cell types of diverse plant species (FIG. 4D). Thus, the manipulation of specific CDPK activities might have important agricultural applications in preventing and protecting crop plants from stress damage and yield loss.

### II. Hormone and Stress Signaling in Protoplasts

Plant hormones, such as auxin are known to activate many early response genes that are likely responsible for diverse aspects of plant growth and development (Davies, P. J., In: Plant hormones, ed., Davies, P. J., pp. 1-12, Kluwer, Dordrecht, Netherlands, 1995; Abel et al., Plant Physiol. 111: 9-17, 1996; Walden et al., Trends Plant Sci. 1: 335-339, 1996). Here we present surprising evidence that a plant MAPK kinase kinase (MAPKKK), NPK1 (Banno et al., Mol. Cell Biol. 13: 4745-4752, 1993), which possesses similar structure to the mammalian TAK1 (Yamaguchi et al., Science 270: 2008-2011, 1995) and fly PK92B (Wassarman et al., Gene 169: 283-284, 1996), activates a MAPK cascade that leads to the repression of early auxin response gene transcription. In addition, we show that a mutation in the kinase domain abolished NPK1 activity, and the presence of the COOH-terminal domain diminished the kinase activity. Moreover, the NPK1 effects on the activation of a MAPK and the repression of early auxin response transcription were specifically eliminated by a MAPK phosphatase (Sun et al., Cell 75: 487-493, 1993). We also found that transgenic tobacco plants overexpressing constitutively active NPK1 produced seeds defective in embryo and endosperm development. These results indicated that auxin sensitivity could be balanced by antagonistical signaling pathways (Bellincampi et al., Plant Cell 8: 477-487, 1996; Coenen et al., Trends Plant Sci. 2: 351-356, 1997) that employ a distinct MAPK cascade in higher plants.

In addition, we provide results showing that constitutively active ANP kinase domains (e.g., ANP1, ANP2, and ANP3) induced the expression of a number of plant stress-inducible gene promoters. Moreover, we provide evidence that transgenic tobacco plants having constitutively active NPK1 produced seedlings that were drought-resistant, as well as resistant to the effects of salt. Such plants were also found to be resistant to other stresses such as heat shock and freezing temperatures.

The examples provided below are for the purpose of illustrating the invention, and should not be construed as limiting.

Auxin Signaling in Maize Leaf Protoplasts

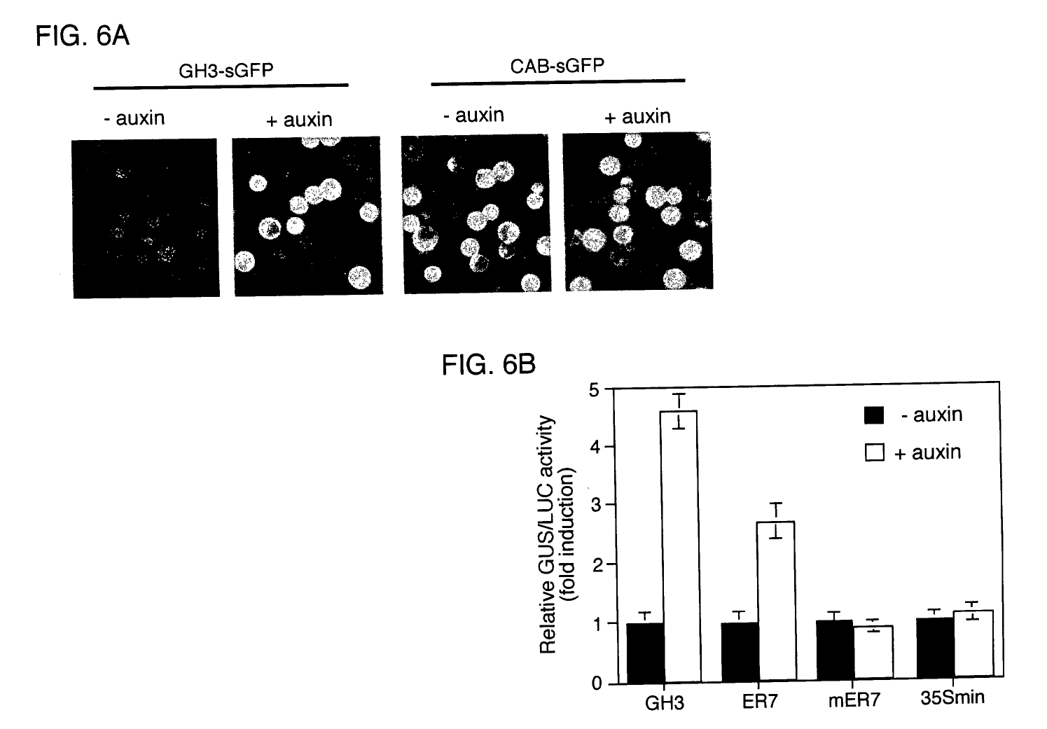

To determine whether the maize transient expression system is suitable for the investigation of auxin signaling, we have tested the auxin inducibility of a well-characterized early response gene promoter, GH3 (Hagen et al., Plant Mol. Biol. 17: 567-579, 1991), in maize mesophyll protoplasts. Maize protoplasts transfected with a construct carrying the coding region of a synthetic green-fluorescent protein (sGFP) (Chiu et al., Curr. Biol. 6: 325-330, 1996) driven by the GH3 promoter (“GH3-sGFP”) showed bright fluorescence upon induction with different active auxin forms, NAA (FIG. 6A) or IAA (data not shown) at 1 mM, a physiologically relevant concentration. In contrast, we observed that auxin did not affect the expression of a GFP construct (“CAB-sGFP”) that was controlled by the maize chlorophyll a/b binding protein gene promoter (CAB5) (Sheen, Plant Cell 2: 1027-1038, 1990) (FIG. 6A).

To confirm the auxin inducibility of the GH3 promoter, we also tested the effect of auxin on the promoter fused to another reporter gene encoding the E. coli β-glucuronidase (GUS) in transfected maize protoplasts. The results from these experiments showed that GUS activity that was controlled by the GH3 promoter was also induced by auxin (FIG. 6B), although the GUS reporter gene generated higher background than the GFP reporter gene in maize cells.

To support the idea that the early auxin responses are conserved in higher plants, we tested an auxin responsive DNA element, ER7 (Ulmasov et al., Science 276: 1865-1868, 1997), which has been found in the majority of early auxin response gene promoters (Abel et al., Plant Physiol 111: 9-17, 1996; Ulmasov et al., supra, 1997). A complementary pair of synthetic oligonucleotides containing the ER7 element was fused upstream of the GUS gene driven by a 35S minimal promoter. This ER7-GUS construct showed auxin inducibility in maize protoplasts, whereas the 35S minimal promoter was found not to be induced by auxin (FIG. 6B). Moreover, when the ER7 element was mutated, it lost its auxin inducibility completely (FIG. 6B), as previously shown in transfected carrot protoplasts (Ulmasov et al., supra, 1997). These data clearly demonstrated that maize mesophyll protoplasts responded to physiological levels of auxin and that the early auxin responses are likely conserved in monocot and dicot plants.

Constitutively Active NPK1 Represses Auxin-Inducible Promoters

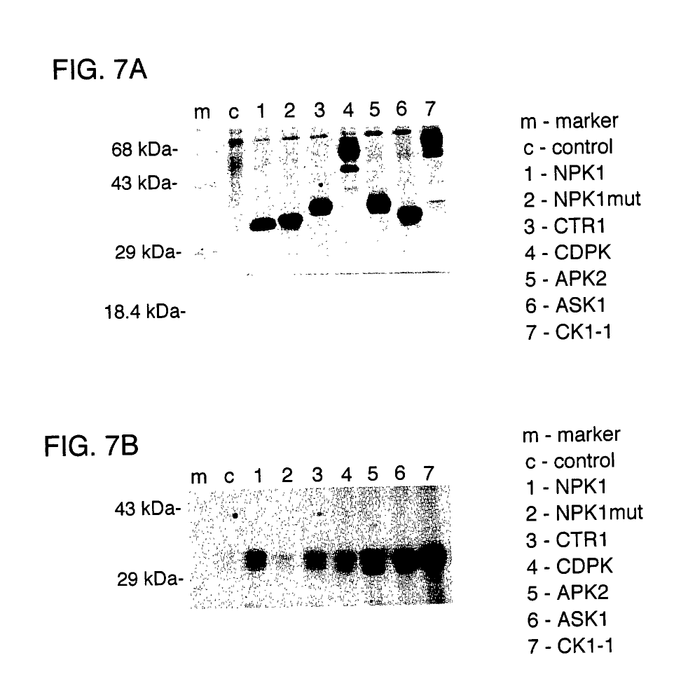

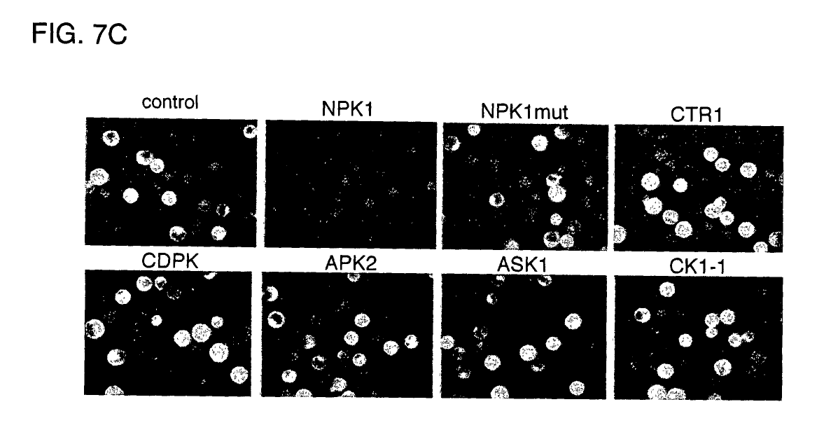

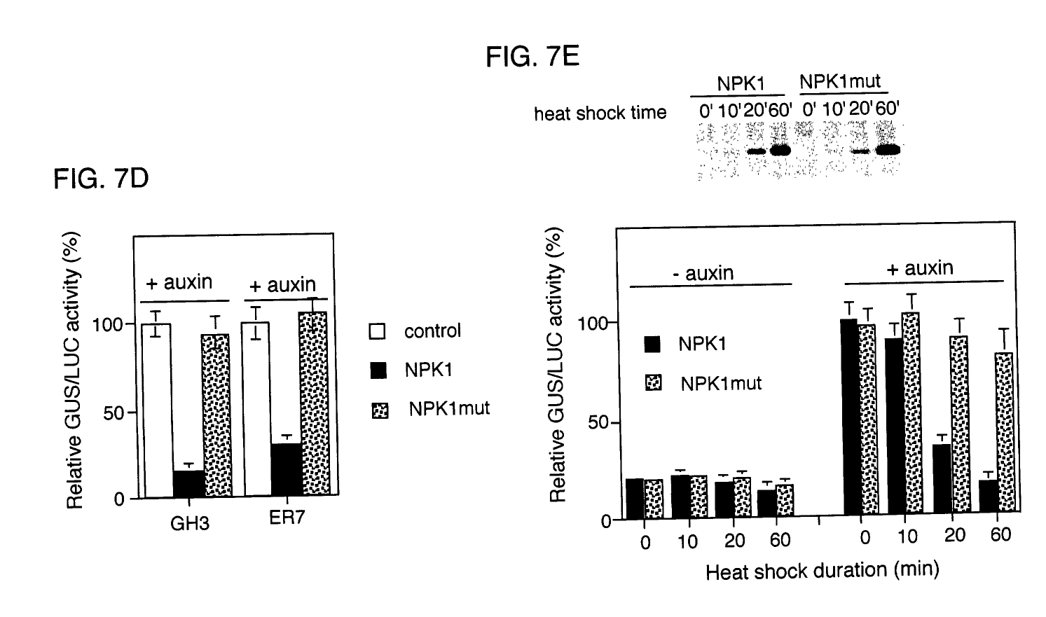

To determine whether NPK1 (Banno et al., supra) is involved in auxin signal transduction, we tested the effect of a constitutively active NPK1 on the activity of the GH3 promoter. It has been shown that MAPKKKs consist of a well-conserved kinase domain and putative regulatory domains. Truncated or naturally occurring MAPKKKs carrying only the kinase domain have been shown to have constitutive kinase activity (Banno, supra; Nishihama et al., Plant J. 12: 39-48, 1997). The structure of NPK1 is unique as a MAPKKK with the kinase domain located at the NH₂-terminus. A similar structure has also been found in the mammalian TAK1 involved in TGF-β signaling (Yamaguchi et al., Science 270: 2008-2011, 1995), and the fly PK92B with an unknown function (Wassarman et al., Gene 169: 283-284, 1996). The kinase domain of NPK1 was tagged with two copies of a hemagglutinin (DHA) epitope (Sheen, supra, 1996) and cloned into a plant expression vector with a derivative of the CaMV35S promoter (this promoter is not affected by auxin) and the nos terminator (Sheen, supra, 1993; Sheen, supra, 1996; Sheen, supra, 1998). The NPK1 construct was co-transfected with the GH3-sGFP or GH3-GUS construct into maize protoplasts. The expression of the NPK1 kinase domain in transfected maize protoplasts was confirmed by ³⁵S-methionine labeling and immunoprecipitation with an anti-HA antibody (FIG. 7A). The kinase activity of the expressed protein was assayed using casein as a universal substrate (FIG. 7B). Surprisingly, the constitutively active NPK1 was found to block auxin activation of the GH3 promoter (FIGS. 7C and 7D).

To show that the kinase activity of NPK1 is necessary for this repression, a null mutation (K109M) was created by site-directed mutagenesis to eliminate the ATP binding site conserved among protein kinases (Sheen, supra, 1996). This mutation was found not to affect the expression of the NPK1 protein (FIG. 7A), but completely abolished the protein kinase activity (FIG. 7B) and the negative effect of NPK1 on the GH3 promoter in the presence of auxin (FIGS. 7C and 7D).

To demonstrate that the inhibitory effect was specific to NPK1, we next tested the effect of another plant MAPKKK, Arabidopsis CTR1, that has been shown to act as a negative regulator of ethylene responses (Kieber et al., Cell 72: 427-441, 1993). The kinase domain of CTR1 was expressed and displayed protein kinase activity in maize protoplasts (FIGS. 7A and 7B), but did not block auxin signaling (FIG. 7C). In addition, because NPK1 is a serine/threonine protein kinase, we expressed other constitutively active serine/threonine protein kinases that belong to four different classes (FIG. 7A), and tested their effect on the GH3 promoter. Unlike NPK1, none of the tested protein kinases repressed the auxin-regulated gene expression (FIG. 7C) although they all exhibited protein kinase activities in the system (FIG. 7B). Thus, the effect of NPK1 on auxin signaling was not due to non-specific phosphorylation in plant cells.

In addition to the GH3 promoter, we examined the effect of the constitutively active NPK1 on the well-established auxin responsive DNA element, ER7, that has been described by Ulmasov et al. (supra, 1997). NPK1 was found to completely suppress the auxin inducibility of the auxin responsive element (FIG. 7D). However, the activities of many auxin-insensitive promoters, including the promoters of CAB, actin, ubiquitin, and CaMV35S genes, were not affected by NPK1 (data not shown). Taken together, these results indicated that NPK1 plays an important and specific role in the negative regulation of the auxin response genes.

It remained possible that NPK1 was a positive regulator in auxin signaling and that the overexpression of NPK1 mimicked the repression of the auxin response genes by very high levels of auxin (Hagen et al., supra). To exclude this possibility, we tested the effect of different NPK1 protein levels on the GH3 promoter activity in the absence or presence of auxin. We used a heat shock promoter (Sheen et al., supra, 1995) to control the amount of the NPK1 protein produced by varying the time of heat shock. The null mutation of NPK1 served as a control for the effect of the heat shock. As is shown in FIG. 7E, the expression levels of the constitutively active NPK1 and the null mutant correlated well with the duration of heat shock. The activation of the GH3 promoter was not observed at any level of NPK1 in the absence of auxin, ruling out the possibility that NPK1 could be a positive regulator in auxin signaling. In the auxin treated protoplasts, the reverse correlation between the NPK1 protein levels and the GH3 promoter activity supports the idea that NPK1 acts as a negative regulator in auxin signal transduction (FIG. 7E).

Analysis of the Putative Regulatory Domains of NPK1

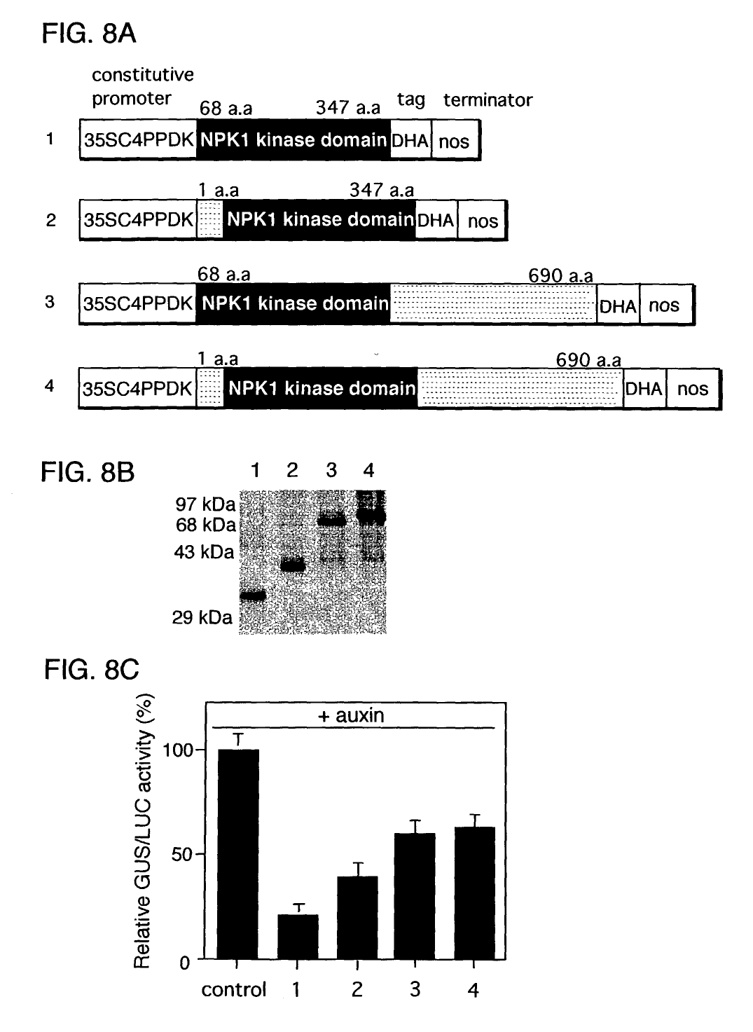

One distinct feature of NPK1 is the presence of a short NH₂-terminal sequence and a long COOH-terminal region outside the kinase catalytic domain (Banno et al., supra). To investigate the function of regions outside the kinase domain in the NPK1 protein, we created several NPK1 deletions (FIG. 8A) and tested their effect on the GH3 promoter activity. Various deletions of the full-length NPK1, as well as the full-length NPK1, showed similar levels of protein expression in transfected maize protoplasts (FIG. 8B). Deletion of the kinase region alone or the kinase domain plus the short NH₂-terminus was found to inhibit the GH3 promoter more strongly than the deletion carrying the kinase domain with the long COOH-terminus or the full-length NPK1 (FIG. 8C).

NPK1 Activates a MAPK

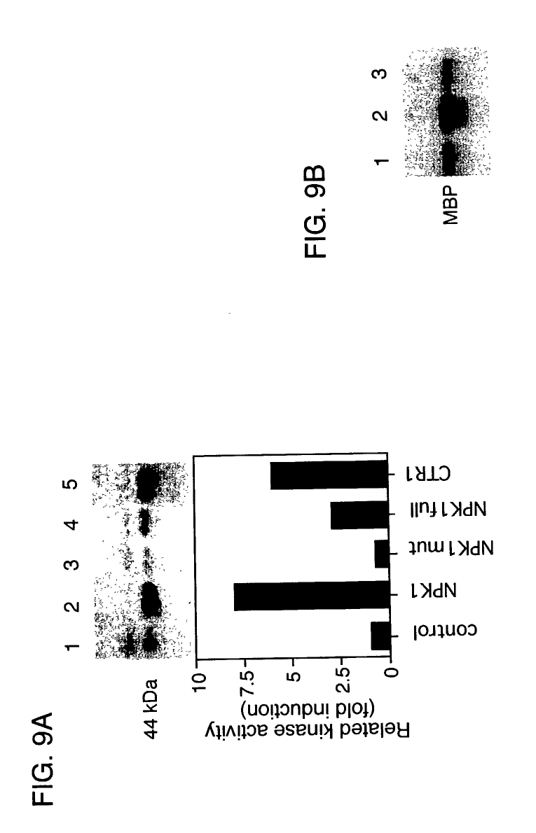

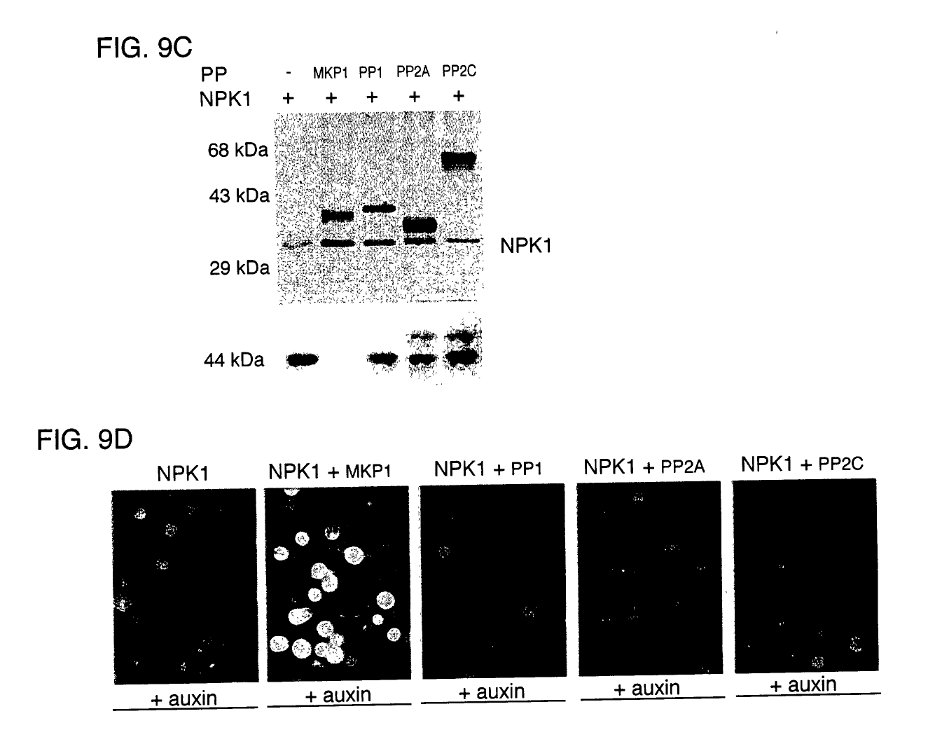

NPK1, as a MAPKKK, is expected to induce a protein phosphorylation cascade resulting in the activation of a MAPK. Although several plant MAPKs have been shown to be induced by stress, hormone, and elicitor signals (Hirt, Trends Biol. Sci. 2: 11-15, 1997; Mizoguchi et al., Trends Biotech. 15: 15-19, 1997), their activation by a phosphorylation cascade has never been demonstrated in plant cells. To determine whether the expression of the constitutively active NPK1 activates an endogenous MAPK in maize protoplasts, we performed a standard MAPK activity assay (Mizoguchi et al., Plant J. 5: 111-122, 1994; Zhang et al., Plant Cell 9: 809-824, 1997; Bogre et al., Plant Cell 9: 75-83, 1997) with extracts prepared from protoplasts transfected with NPK1 using myelin basic protein (MBP) as a substrate. As shown in FIG. 9A, protoplasts which were transfected with the constitutively active NPK1 had about eight-fold higher 44 kDa kinase activity than protoplasts transfected with the NPK1 null mutation or plasmid DNA carrying no plant genes. This result suggested that the expression of the constitutively active NPK1 resulted in activation of a MAPK. Apparently, a MAPKK was already present in maize protoplasts and sufficient to relay phosphorylation from MAPKKK (NPK1) to the 44 kDa MAPK. The expression of the full-length NPK1 increased the putative MAPK activity only three-fold (FIG. 9A). These results are consistent with the observation that the full-length NPK1 has less effect and the null NPK1 protein has no effect on the repression of the GH3 promoter in the presence of auxin (FIGS. 7C, 7D, and 7E; FIG. 8C). As a control, the constitutively active CTR1 also activated an endogenous kinase (FIG. 9A), suggesting the existence of another unrelated MAPK cascade in maize protoplasts. We also observed that the constitutively active CTR1, but not NPK1, could repress ethylene responsive GCC1 enhancer activity more than ten-fold in Arabidopsis protoplasts, consistent with the proposed role of CTR1 as a negative regulator in the ethylene signaling pathway (Kieber et al., Cell 72: 427-441, 1993; Sheen, unpublished).

To verify that NPK1 expression resulted in the activation of a MAPK, we performed kinase activity assays with the proteins immunoprecipitated with an antibody raised against two conserved domains of a mammalian MAPK. The MAPK activity of the protoplasts transfected with the constitutively active NPK1 was significantly higher than that of the cells transfected with the NPK1 null mutant (FIG. 9B). These data are consistent with the results of the MAPK in-gel assay (FIG. 9A), and demonstrate that tobacco NPK1 can induce a kinase cascade in maize protoplasts that activates an endogenous maize MAPK.

To determine whether the 44 kDa MAPK is involved in the repression of early auxin response genes, we tested the effect of a specific MAPK-phosphatase (MKP) that can inactivate MAPKs. Protein phosphatases that can specifically dephosphorylate/inactivate MAPKs have been reported in a variety of eukaryotes and are evolutionarily conserved (Tonks et al., Cell 87: 365-368, 1996). A mouse MKP1 (Sun et al., supra), highly specific to MAPKs, was cloned into the plant expression vector and expressed in maize protoplasts (FIG. 9C). The expression of MKP1 resulted in the complete elimination of the NPK1 effects, including the NPK1-dependent activation of a MAPK (FIG. 9C) and the repression of the auxin-inducibility of the GH3 promoter (FIG. 9D). The results suggest that the activation of the 44 kDa MAPK is necessary for the NPK1 dependent repression of transcription. As controls, the expression of other plant protein phosphatases (PP) that belong to the three serine/threonine classes, PP1, PP2A, and PP2C, did not abolish the activation of MAPK by NPK1 (FIG. 9C) or the repression of the GH3 promoter by NPK1 (FIG. 9D), despite the detection of enhanced PP activities in transfected maize protoplasts (Sheen, supra, 1993; Sheen, supra, 1998) (data not shown). The fact that MKP1 alone does not affect the GH3 promoter (data not shown) supports our current model that a signal(s), antagonizing auxin responses, induces NPK 1-like MAPKKKs and leads to the repression of the auxin-inducible transcription.

Stress and Auxin Responses in Arabidopsis Protoplasts

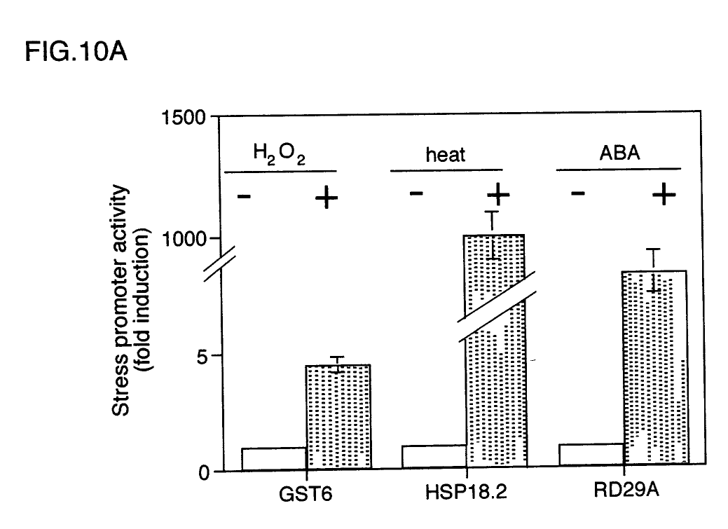

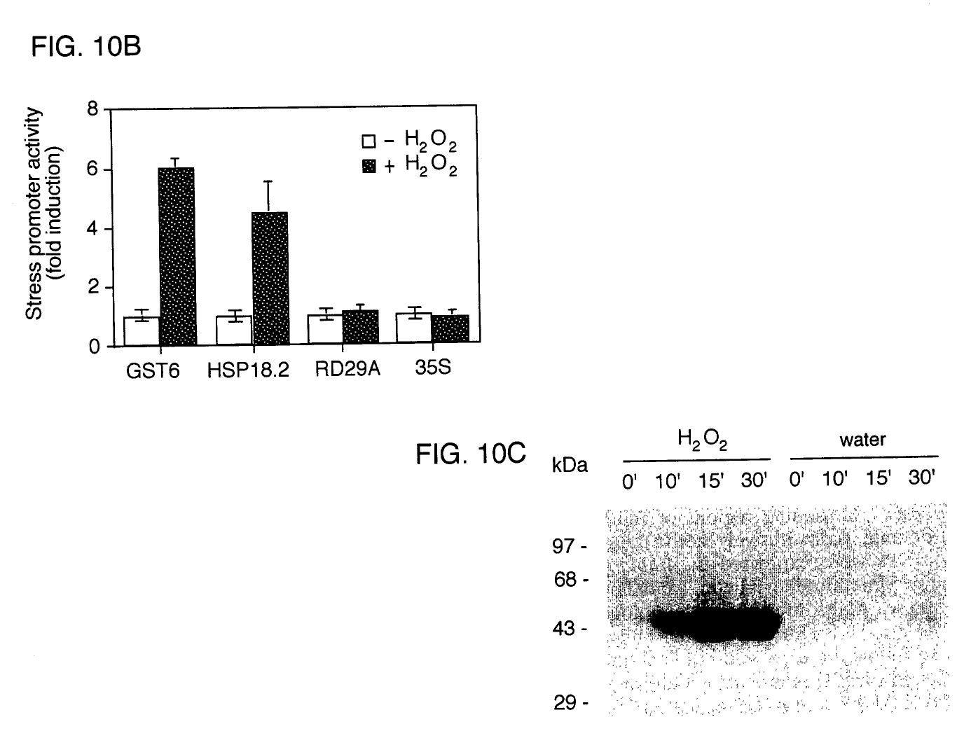

To further elucidate the molecular basis of oxidative stress signaling in plants, we have also showed that an Arabidopsis protoplast transient expression system is useful to investigate multiple stress responses. Three Arabidopsis stress responsive promoters, glutathione S-transferase GST6 (Chen et al., Plant J. 10: 995-966, 1996), heat shock HSP18.2 (Takahashi and Komeda, Mol. Gen. Genet. 219: 365-372, 1989), and the abscisic acid (ABA) responsive promoter RD29A (Yamaguchi-Shinozaki et al., Plant Physiol. 101: 1119-1120, 1993; Ishitani et al., Plant Cell 9: 1935-1949, 1997), were fused to the luciferase (LUC) reporter and tested for their responses in transfected mesophyll protoplasts. The GST6, HSP18.2, and RD29A promoters were activated by H₂O₂, heat, and ABA, respectively, in protoplasts (FIG. 10A) as demonstrated previously in intact plants (Chen et al., supra; Takahashi and Komeda, supra; Yamaguchi-Shinozaki et al., supra; Ishitani et al., supra). Several GST genes, including GST6, have been shown to be induced by high and toxic concentrations of plant growth hormone auxin, as well as by physiologically inactive auxin analogs, heavy metals, and numerous stresses (Chen et al., supra; Ulmasov et al., Plant Mol. Biol. 26: 1055-1064, 1994; Abel and Theologis, Plant Physiol. 111: 9-17, 1996; Sitbon and Perrot-Rechenmann, Physiol. Plantarum 100: 443-455, 1997; Guilfoyle et al., Plant Physiol., 118: 341-347, 1998, Marrs, Annu. Rev. Plant Physiol. Plant Mol. Biol. 47: 127-158, 1996). This non-specific induction of GSTs separates them from other auxin responsive genes that are only induced by low physiological levels of active auxin, and indicates that stress rather than auxin is responsible for the activation of the GST genes.

H₂O₂ Activates Oxidative Stress Inducible Promoters and Two MAPK-Like Kinases in Arabidopsis Protoplasts

Oxidative stress signal transduction was also examined by independently fusing three Arabidopsis stress responsive promoters, GST6, HSP18.2, and RD29A, that are activated by oxidative stress, heat shock, and ABA/drought/cold, respectively, to the LUC reporter and tested for their responses in transfected mesophyll protoplasts. Oxidative stress (H₂O₂) activated the GST6 and the heat shock promoters in the transfected protoplasts (FIG. 10B). The results were similar to those previously reported for other systems. However, the ABA/drought/cold inducible RD29A promoter was not affected by H₂O₂. The RD29A promoter was functional in the transfected protoplasts since ABA could induce the promoter (as discussed infra). The H₂O₂ treatment also had no significant effects on UBQ10 promoter activity, which served as an internal control, or on the CaMV35S promoter activity (FIG. 10B).

To determine whether H₂O₂ signaling is mediated through an evolutionarily conserved MAPK cascade, we performed a MAPK activity in-gel assay with extract from protoplasts challenged with H₂O₂. Within ten minutes, treatment with H₂O₂ but not with water, activated two myelin basic protein (MBP, a common MAPK substrate) phosphorylation activities (FIG. 10C).

H₂O₂ and Heat Shock Suppress the Auxin Responsive GH3 Promoter

H₂O₂, heat, and ABA can arrest cell cycle and plant growth (Inzé and Van Montagu, supra; Bolwell and Wojtaszek, supra; Lamb and Dixon, supra; Noctor and Foyer, supra; Leung et al., supra; Cheikh and Jones, Plant Physiol. 106, 45-51, 1994; Reichheld et al., Plant J. 17: 647-656, 1999), the processes promoted by auxin (Key, BioEssays 11: 52-58, 1989; Garbers and Simmons, Trend Cell Biol. 4: 245-250, 1994; Walker and Estelle, Curr. Opinion Plant Biol. 1: 434-439, 1998; Leyser, Curr. Biol. 8: R305-R307, 1998). This suggests a connection between stress and auxin signaling; however, a molecular basis of the crosstalk is unknown. We tested the effects of these stresses on the activity of the auxin responsive promoter, GH3 (Hagen et al., supra; Liu et al., supra). In Arabidopsis protoplasts, physiological concentrations of auxin, 1 PM NAA (FIG. 10D) or 1 μM IAA (data not shown), dramatically increased GH3 promoter activity. The kinetics and magnitude of GH3 promoter activation in Arabidopsis protoplasts were comparable to those previously reported in other systems (Hagen et al., supra; Liu et al., supra). Both H₂O₂ and heat, but not ABA, severely abolished the auxin response (FIG. 10D). The same stress treatments had no significant effects on the CaMV35S promoter activity as an internal control or on ubiquitin promoter UBQ10 activity as a parallel control (data not shown). The repression of the auxin early response gene promoter is therefore likely due to the activation of a specific stress signaling pathway that is common to H₂O₂ and heat, two representative oxidative stress signals (Inze and Van Montagu, supra; Bolwell and Wojtaszek, supra; Lamb and Dixon, supra; Noctor and Foyer, supra). In contrast, the stress hormone ABA did not appear to interfere with auxin signaling in leaf cells.

ANP 1 Initiates a Stress MAPK Cascade

In many eukaryotes, the transduction of H₂O₂ and heat stress signals is controlled by protein phosphorylation involving MAPKs (Kyriakis and Avruch, J. Biol. Chem. 271: 24313-24316, 1996; Tuomainan et al., Plant J. 12: 1151-1162, 1997; Gustin et al., Microbiol. Mol. Biol. Review 62: 1264-1300, 1998; Morimoto, Genes Dev. 12: 3788-3796, 1998; Morimoto and Santoro, Nature BioTech. 16: 833-838, 1998; Schoffl et al., Plant Physiol. 117: 1135-1141, 1998). MAPK and immediate upstream activators, MAPKK and MAPKKK, constitute a functionally interlinked MAPK cascade (Kyriakis and Avruch, supra; Tuomainan et al., supra; Gustin et al., supra). Since the activated tobacco MAPKKK, NPK1 (Banno et al., supra), initiated a MAPK cascade that represses auxin early response gene expression (as described herein), we reasoned that this MAPK cascade could also represent a molecular link between oxidative stress and auxin signal transduction. Three Arabidopsis NPK1-like MAPKKKs, ANP1-3, share high homology in both their kinase and regulatory domains (Nishihama et al., Plant J. 12: 39-48, 1997). The regulatory domains of MAPKKKs interact mostly with upstream regulators, whereas the kinase domain forms a complex with the substrate, a specific MAPKK (Xu et al., Proc. Natl. Acad. Sci. USA 92: 6808-6812, 1995; Shibuya et al., Science 272: 1179-1182, 1996; Clark et al., Proc. Natl. Acad. Sci. USA 95: 5401-5406, 1998; Ichimura et al., Biochem. Biophys. Res. Comm. 253: 532-543, 1998; Posas and Saito, EMBO J. 17: 1385-1394, 1998; Saitoh et al., EMBO J. 17: 2596-2606, 1998; Xia et al., Genes Develop. 12: 3369-3381, 1998; Yuasa et al., J. Biol. Chem. 273: 22681-22692, 1998). Deletions of the regulatory domains, as a result of genetic manipulations, naturally occurred alternative splicing, or proteolytic cleavage, increase MAPKKK activity (Banno et al., supra; Xu et al., supra, 1995; Shibuya et al., supra, 1996; Clark et al., supra; Ichimura et al., supra. 253: 532-543, 1998; Posas and Saito, supra; Saitoh et al., supra; Xia et al., supra; Yuasa et al., supra; Deak et al., supra).

ANPs Activate Two Endogenous MAPKs

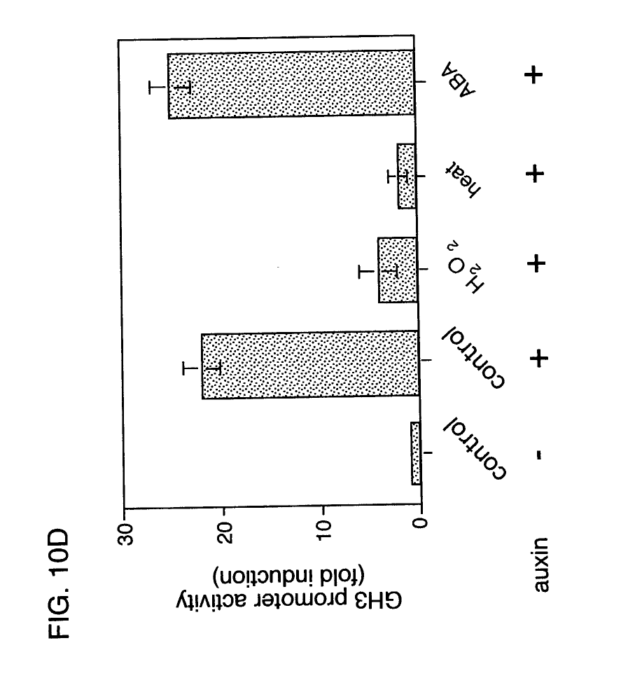

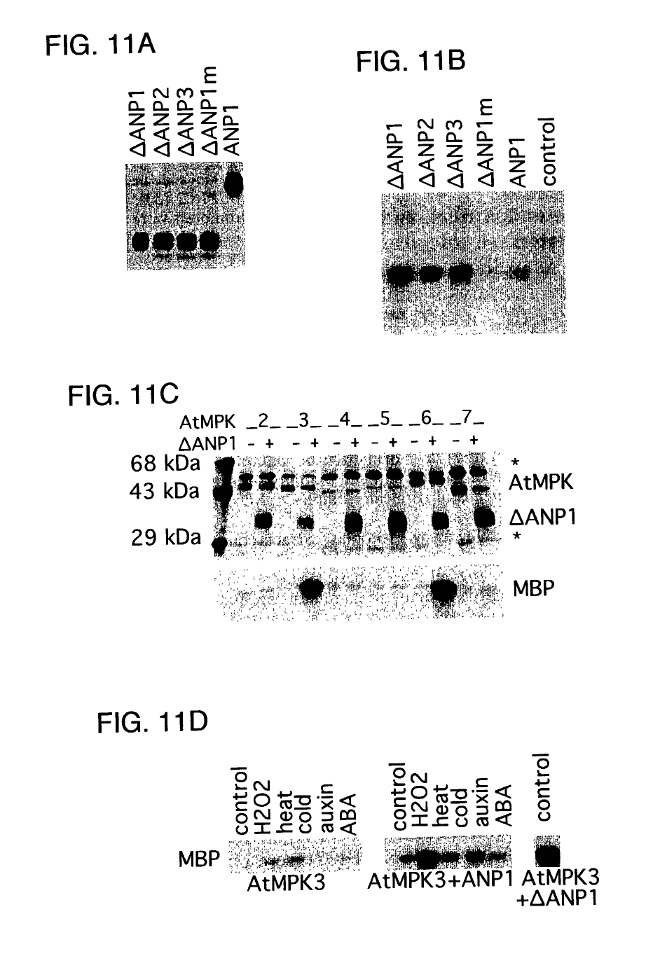

We first verified that ANPs could activate endogenous MAPKs in Arabidopsis. Coding regions of full length (repressed), kinase domain (constitutively active), or mutated (kinase-inactive) ANPs were fused to the haemagglutinin (HA) epitope tag and expressed in Arabidopsis protoplasts (FIG. 11A).

Constitutively active ANPs activated two putative endogenous MAPKs in transfected protoplasts (FIG. 11B). Moreover, a mutation in the ATP binding site abolished, and the presence of the regulatory domains diminished, the ability of ANP1 to activate the putative MAPKs. The sizes of the ANP-activated kinases are similar to those reported for plant MAPKs (Hirt, Trends Biol. Sci. 2: 11-15, 1997; Machida et al., Critic Rev. Plant Sciences 16: 481-496, 1997; Zhang and Klessig, Plant Cell 9: 809-824, 1997; Mizoguchi et al., Trends BioTech. 15: 15-19, 1997; Jonak et al., Cell Mol. Life Sci. 55: 204-213,1999).

ANPs1 Induce AtMKP3 and AtMPK6 In Vivo

To identify downstream MAPKs of the ANP-mediated MAPK cascade, constitutively active ANP1 was co-transfected with one of six Arabidopsis MAPKs (AtMPKs), representing three different classes (Hirt, supra; Machida et al., supra; Zhang and Klessig, supra; Mizoguchi et al., supra; Jonak et al., supra). The active ANP1 initiated a MAPK cascade that could be assayed by measuring the activity of an individual epitope-tagged AtMPK after immunoprecipitation (FIG. 11C). Constitutively active ANP1 slightly changed the mobility of AtMPK3 and AtMPK6 detected by SDS-PAGE, suggesting phosphorylation of these MAPKs (FIG. 11C, upper panel). Notably, active ANP1 dramatically increased the activity of only these two MAPKs (FIG. 11C, lower panel). Active ANP2 and ANP3, but not another MAPKKK, CTR1 (Kieber et al., Cell 72: 427-441, 1993), also induced AtMPK3 and AtMPK6 activity (data not shown), indicating that CTR1 and ANPs activate different MAPK cascades. AtMPK3 and AtMPK6 belong to the class of MAPKs implicated in both stress and pathogen signal transduction in many different plant species (Hirt, supra; Machida et al., supra; Zhang and Klessig, supra; Mizoguchi et al., supra; Jonak et al., supra). The ability of ANPs to activate stress-related MAPKs indicates that an ANP-mediated MAPK cascade is involved in stress signaling.

Stresses Activate AtMKP3 and ANP1

To define the stress signals that can regulate the MAPK cascade, HA epitope-tagged AtMPK3 was transfected into Arabidopsis protoplasts, and the protoplasts were then challenged with different stresses. Phosphorylation activity of AtMPK3 was measured after immunoprecipitation with an anti-HA antibody. Several stress signals, including H₂O₂ or heat, but not auxin, activated AtMPK3 (FIG. 11D, left). H₂O₂ or heat also activated AtMPK6 (data not shown). However, when the full-length ANP1 protein was ectopically expressed, only these two stresses, but not other stress stimuli, could further enhance the activation of AtMPK3 (FIG. 11D, center). The fact that H₂O₂ and heat each induced the full-length ANP 1 activity to the level of the constitutively active ANP1 (FIG. 11D, right) indicates that ANP1 functions in mediating H₂O₂ and heat stress signal transduction. Induction of AtMPK3 by stimuli unrelated to oxidative stress is probably mediated by an ANP-independent pathway (FIG. 11D, left).

ANP 1 Activates Stress-Inducible Promoters

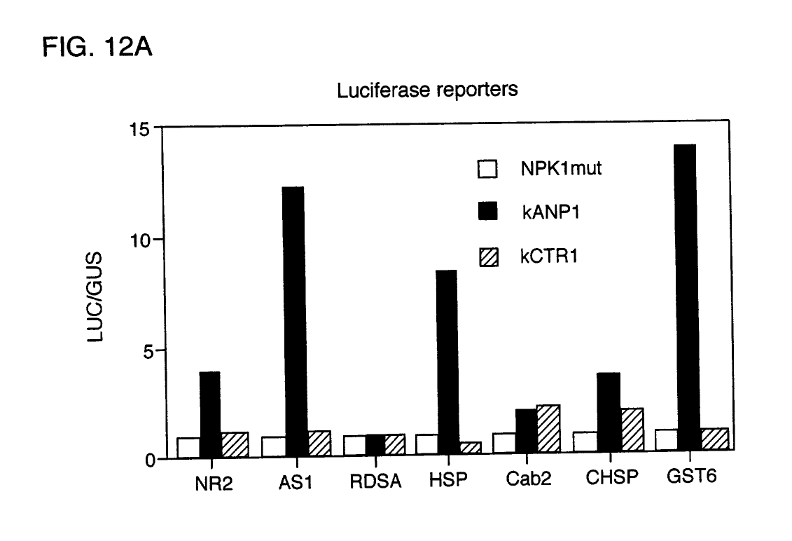

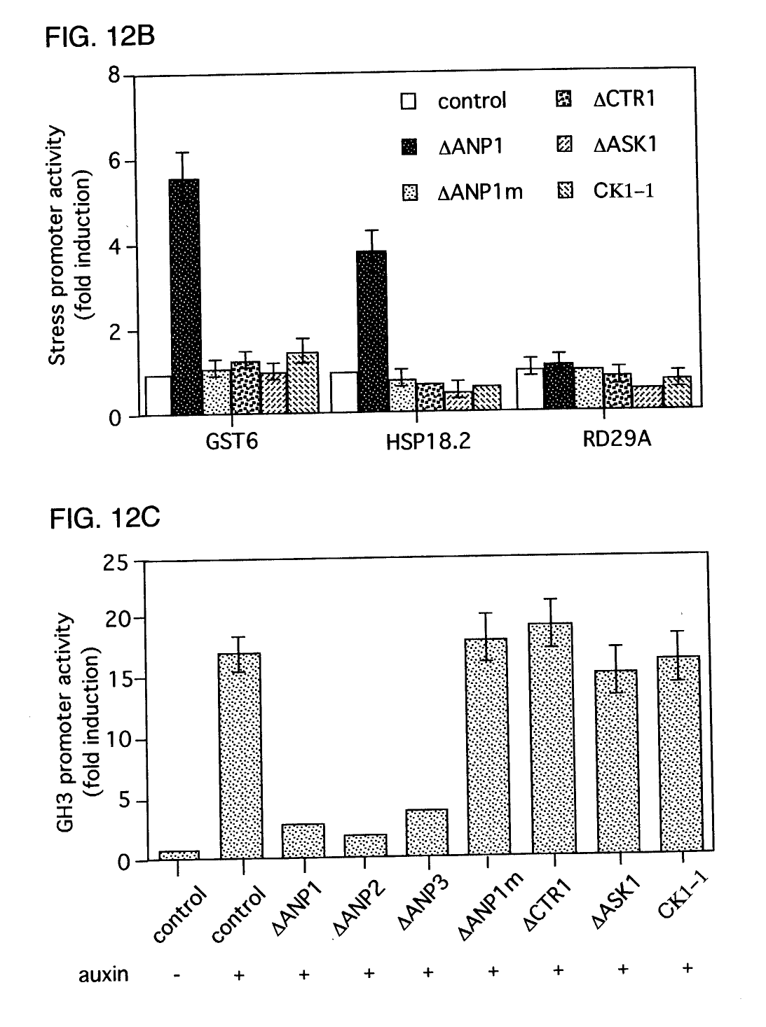

To determine whether a plant MAPKKK, such as ANP1 (Nishihama et al. Plant J. 12: 39-48, 1997), is involved in stress signal transduction, we have tested the effect of a constitutively active ANP1 kinase domain on the activity of several different dicot promoters. This was achieved by introducing into Arabidopsis protoplasts a transgene construct consisting of the firefly luciferase coding sequence (LUC) under the control of different dicot promoters. The promoters tested were the nitrate reductase, NR2, promoter from Arabidopsis (Lin et al., Plant Physiol. 106: 477-484, 1994); the asparagine synthetase, AS1, promoter (Neuhaus et al., EMBO J. 16: 2554-2564, 1997); the RD29A Arabidopsis stress-responsive promoter (Ishitani et al., Plant Cell 9: 1935-1949, 1997); the Arabidopsis HSP heat shock promoter (Sheen et al., Plant Journal 9: 777-784, 1995; Takahashi et al., Plant J. 2: 751-761, 1992); the Cab2 promoter (Mitra et al. Plant Mol. Biol. 12: 169-179, 1989); the chalcone synthase gene promoter (Feinbaum et al., Mol. Cell Biol. 8: 1985-1992, 1988); and the H₂O₂-inducible glutathione S-transferase promoter (GST) from Arabidopsis (Chen et al., Plant J. 10: 955-966, 1996). The kinase domain of ANP1 was cloned into a plant expression vector with a derivative of the 35S promoter and the nos terminator (Sheen, Science 274: 1900-1902, 1996). The ANP1 construct was co-transfected with one of the dicot promoter reporter gene construct and assayed according to standard methods. Surprisingly, the constitutively active ANP1 kinase domain was found to activate the expression of the AS1, HSP, and GST6 promoters (FIG. 12A). Constitutive expression of either the mutated NPK1 kinase domain or the CTR1 kinase domain had no effect on the expression of the dicot reporter genes.

To provide further evidence for the involvement of ANPs in specific stress signaling, we tested the effect of the constitutively active ANP 1 on the activity of the GST6, HSP18.2, and RD29A promoters. The active ANP1 could substitute for H₂O₂ and heat to induce the GST6 and HSP 18.2 promoters respectively, but did not change the expression of the ABA, cold, or drought responsive RD29A promoter (FIG. 12B). The activation of the GST6 and HSP18.2 promoters required ANP kinase activity since a single amino acid mutation in the ATP binding site completely abolished the ANP 1 effect on the promoters. However, the activation was not due to non-specific protein phosphorylation because three other Arabidopsis protein kinases, including another constitutively active MAPKKK, CTR1 (Kieber et al., supra), did not affect the promoters' activities. The tested protein kinases were expressed equally well and were at least as active as ANP-like MAPKKKs in transfected cells (as described herein). These results reinforce a role of ANP 1 in H₂O₂ and heat signal transduction. However, since ANP 1-mediated induction of the HSP18.2 promoter was lower than that obtained by heat shock (FIG. 10A), both ANP-dependent and ANP-independent pathways are probably required to fully activate the heat shock promoter. Since oxidative stress can induce heat shock responsive genes (Morimoto, supra; Morimoto and Santoro, supra; Schoffl et al., supra; Banzet et al., Plant J. 13: 519-527, 1998; Storozhenko et al., Plant Physiol. 118: 1005-1014, 1998; Zhong et al., Mol. Cell 2: 101-108, 1998; Landry and Huot, Biochem Soc. Symp. 64: 79-89, 1999), active oxygen species generated by heat shock (Inze and Van Montagu, supra; Bolwell and Wojtaszek, supra; Lamb and Dixon, supra; Noctor and Foyer, supra) might be responsible for ANP-dependent activation of the promoter.

ANPs Repress the Auxin Response

To determine whether ANPs can mimic H₂O₂ and heat to repress auxin signaling, we tested the effect of the kinases on GH3 promoter activity. Constitutively active ANP1, ANP2, and ANP3, but not other tested protein kinases, effectively suppressed the GH3 promoter induction by auxin (FIG. 12C). The results suggest that Arabidopsis ANPs are orthologs of the tobacco NPK1 that can suppress auxin signaling (as described herein). Thus, similar to H₂O₂ and heat, the constitutively active ANPs can repress the auxin inducible promoter and induce expression of the GST and HSP genes (FIGS. 10A, 10D and FIGS. 12B, 12C).

Because H₂O₂ can activate the ANP-mediated MAPK cascade, we reasoned that oxidative stress would be able to repress the GH3 promoter activity. Indeed, H₂O₂ abolished the auxin response without affecting the internal control UBQ10-GUS and the activity of 35S-LUC. In contrast, the stress hormone ABA, that can activate the RD29A promoter in the system, did not appear to interfere with auxin signaling in leaf cells. As H₂O₂ can arrest the cell cycle, while auxin promotes the process, there may be shared mechanisms in oxidative stress and auxin signaling. Our finding that the H₂O₂-induced MAPK cascade can repress auxin responses provides the first molecular link between oxidative stress and auxin signal transduction. The ANP-mediated MAPK cascade may help stressed plants to shift energy from auxin-dependent activities to stress protection and survival.

Analyses of Transgenic Tobacco Plants Expressing NPK1

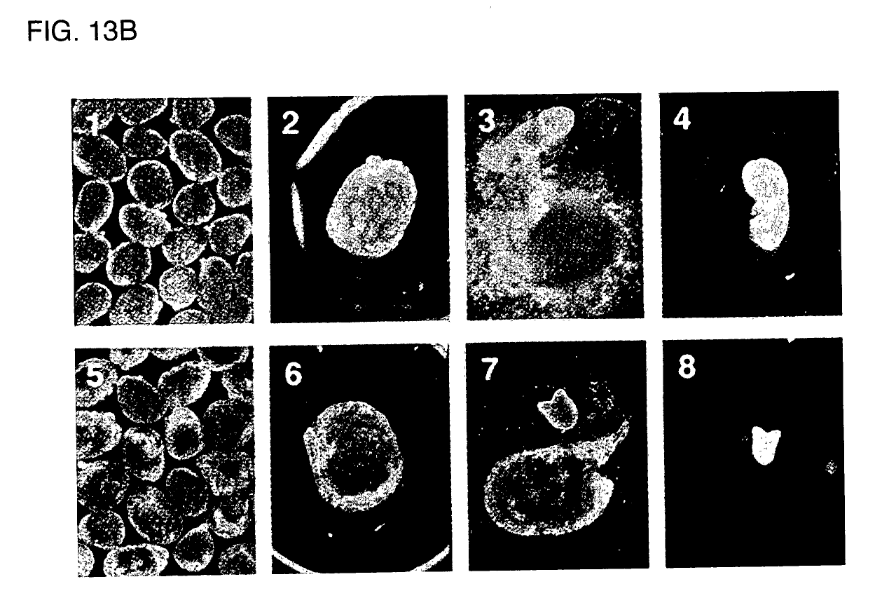

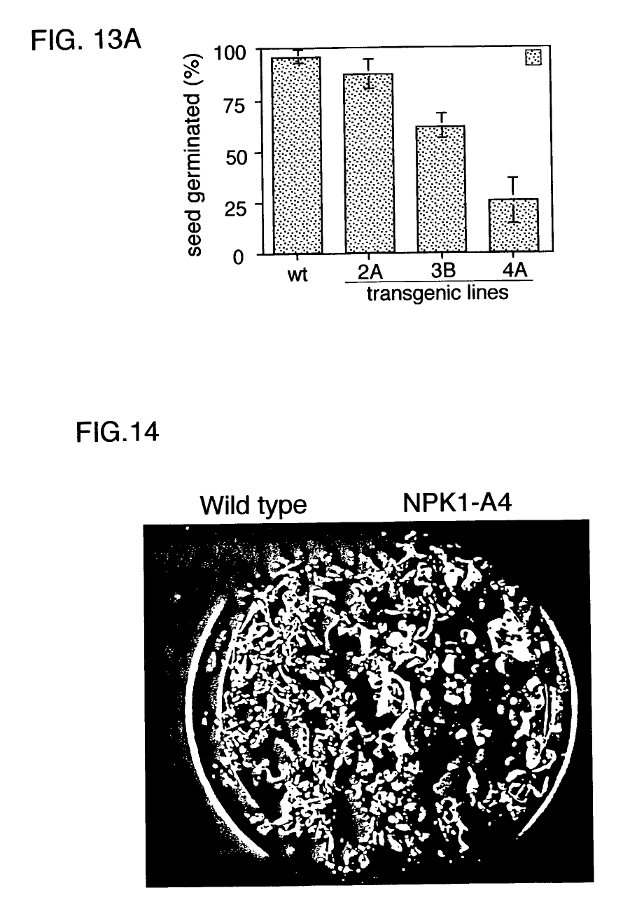

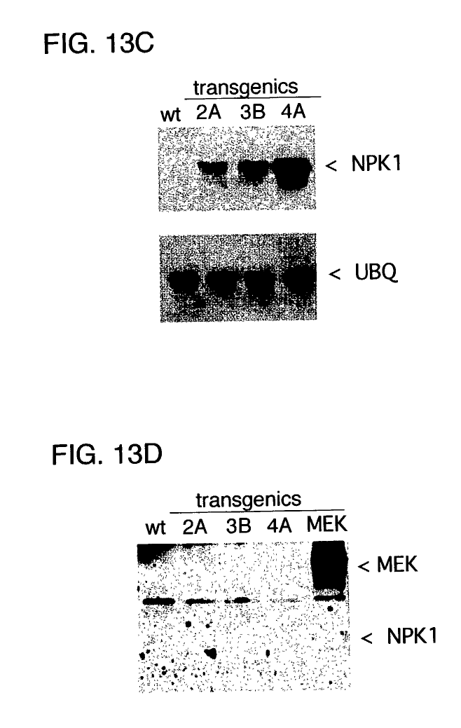

To assess the function of NPK1 at a whole plant level, we generated transgenic tobacco plants ectopically overexpressing the constitutively active NPK1. It was anticipated that overexpression of NPK1, as an auxin antagonist, could be lethal. We obtained transgenic plants through three independent transformation experiments. We observed that some seeds from several independent NPK1 transgenic lines never germinated, whereas seeds from the wild-type control (FIG. 13A) and many other tobacco lines carrying other transgenes (data not shown) germinated normally. In one line, designated 4A, more than 75% of the seeds did not germinate under any conditions. A closer examination revealed that some transgenic seeds exhibited underdeveloped embryo and endosperm (FIG. 13B). Importantly, the number of defective seeds in each line correlated with the level of transgene expression (FIG. 13D), suggesting that the seed phenotype was due to transgene expression. Although the majority of the transgenic seeds that survived expressed the NPK1 mRNA (FIG. 13C), they produced mostly wild-type looking plants. However, we could not detect the ectopic HA-tagged NPK1 protein in these normal-looking transgenic plants after numerous protein blot analyses, while the control transgenic line expressing the HA-tagged MEK1 showed a strong signal (FIG. 13D). We hypothesize that the truncated NPK1 protein is unstable and cannot accumulate to a level required for causing grossly abnormal growth. This is in agreement with a recent report that a mammalian MAPKKK MEKK1 is degraded rapidly after processing and activation (Widmann et al., Mol. Cell. Biol. 18: 2416-2429, 1998). In addition, it was reported that in tobacco cells the NPK1 protein is subjected to a fast turn-over after activation specifically at the end of M phase in the cell cycle (Machida et al., 40th NIBB Conference “Stress responses”, 1998), and is detectable at low levels only in fast-growing tissues (Banno et al., supra). Thus, accumulation of NPK1 protein might be tightly regulated in plants. This likely explains why the most dramatic effect of NPK1 during embryogenesis and seed development were observed when rapid cell division occurs and more NPK1 proteins may accumulate to block cell cycle progress. The auxin requirement for embryogenesis in plants has been demonstrated (Mordhorst et al., Genetics 149: 549-563 1998). Similarly, ectopic activation of a MAPK cascade disrupts Xenopus embryo development by inducing mitotic arrest specifically at the M phase (Takenaka et al., Science 280: 599-602, 1998).

Transgenic Tobacco Plants Expressing NPK1 Are Resistant to Drought and Excess Salt

Transgenic tobacco plants overexpressing the constitutively active NPK1 were found to be resistant to limited water availability when compared to non-transgenic plants (FIG. 14). In addition, transgenic tobacco seeds constitutively expressing the NPK1 gene were also observed to germinate and grow under high salt conditions (150 mM NaCl), as well as to thrive after exposure to oxidative and heat stresses.

Stress Tolerance of Transgenic Tobacco Plants Ectopically Expressing Active NPK1

GSTs and HSPs encode conjugation enzymes and molecular chaperones, respectively. They play essential roles in detoxification and stabilization of damaged proteins and assisting cell recovery from stresses (Marrs, supra; Morimoto, supra; Morimoto and Santoro, supra; Schoffl et al., supra). Constitutive expression of GSTs or HSPs in transgenic tobacco and Arabidopsis can make plants more resistant to different stresses, such as salt and heat (Tarczynski et al., Science 259: 508-510, 1993; Kishor et al. Plant Physiol. 108: 1387-1394, 1995; Lee et al. Plant J. 8: 603-612, 1995; Ishizaki-Nishizawa et al., Nature BioTech. 14: 1003-1006, 1996; Roxas et al., Nature BioTech. 15: 988-991, 1997; Prandl et al., Mol. Gen. Genet. 258: 269-278, 1998; Jaglo-Ottosen et al., Science 280: 104-106, 1998; Liu et al. Plant Cell 10: 1391-1406, 1998; Pardo et al., Proc. Natl. Acad. Sci. USA 95: 9681-9686, 1998; Pei et al., Science 282: 287-290, 1998). Since constitutively active ANP1 induces expression of GST6 and HSP18.2 (FIG. 7B), it is possible that transgenic plants ectopically expressing the active ANP-like protein might be more tolerant to such stresses.

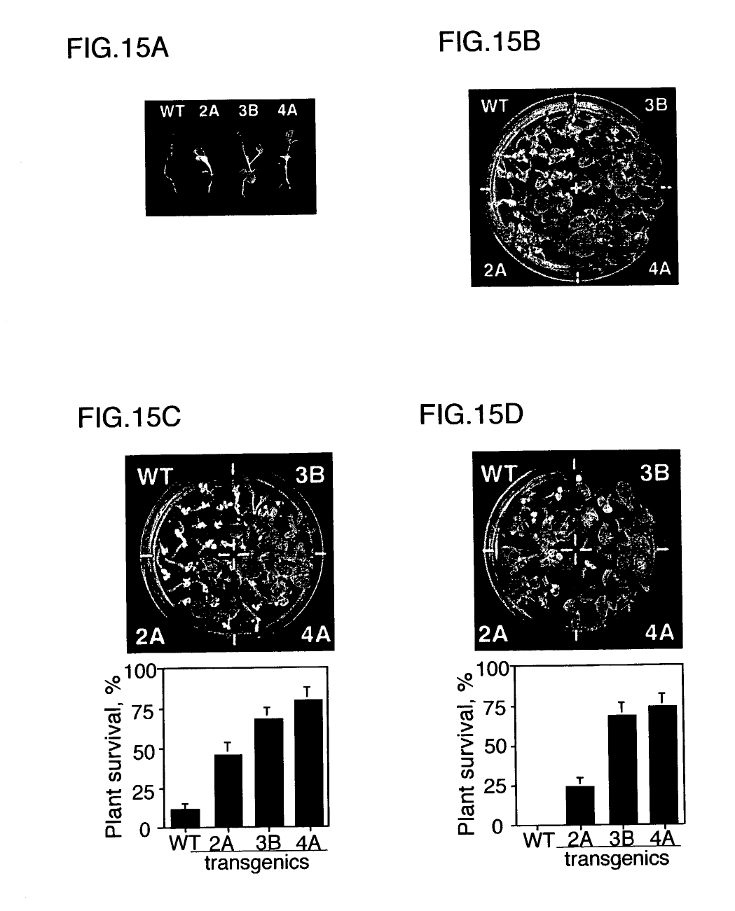

Several transgenic tobacco lines (2A, 3B, 4A), expressing different levels of the constitutively active tobacco ANP ortholog, NPK1 (as described herein), were examined. Phenotypically, the transgenic plant did not differ from wild-type plants under normal growth conditions (FIG. 15A). However, transgenic plants grew more vigorously than did the wild-type plants in the presence of 150 mM NaCl. In addition, only 12% of the wild-type, but 46%, 68%, and 80% of 2A, 3B, and 4A plants, respectively, survived a three-day exposure to high salt (300 mM NaCl) (FIG. 15C). NPK1 Transgenic plants were also observed to be tolerant to a 3 hour freezing temperature treatment of −10° C. (FIG. 15B). We have also tested the sensitivity of NPK1 transgenic plants to heat shock. Exposure to 48° C. heat shock killed all the wild-type plants, but 24% of 2A, 68% of 3B, and 74% of 4A plants survived (FIG. 15D). The stress tolerance of these NPK1 transgenic plants was proportional to the level of NPK1 transgene expression (as discussed herein). Thus, similar to tobacco and Arabidopsis overproducing GSTs and HSPs (Tarczynski et al., supra; Kishor et al. supra; Lee et al., supra; Ishizaki-Nishizawa et al., supra; Roxas et al., supra; Prandl et al., supra; Jaglo-Ottosen et al., supra; Liu et al., supra; Pardo et al., supra; Pei et al., supra), the NPK1 transgenic plants were more tolerant to salt and heat than were wild-type plants. Although some of the NPK1 transgenic seeds are defective during embryogenesis (as discussed herein) when auxin signaling plays a crucial role (Michalczuk et al., Phytochem. 31: 1097-1103, 1992; Ribnicky et al., Plant Physiol. 112: 549-558, 1996; Hardtke and Berleth, EMBO J. 17: 1405-1411, 1998; Mordhorst et al., Genetics 149: 549, 1998; McGovern et al., 9^{th} Arabidopsis Conference, Madison, USA 1998), the absence of obvious growth defects in post-embryonic development of the transgenic plants suggests that the level of NPK1 expression achieved is not deleterious, but rather beneficial in vegetative tissues. The manipulation of this oxidative stress signaling regulator can protect plant cells from diverse environmental stresses, such as heat and high salt. This approach may even be applied for protection from other environmental stresses, such as UV-B, ozone, photooxidation, herbicide, pathogen, drought, and chilling that also involve oxidative stress damage (Green and Fluhr, Plant Cell 7: 203-212, 1995; Prasad, Plant J. 10: 1017-1026, 1996; Willekens et al., EMBO J. 16: 4806-4816, 1997; Chamnongpol et al., Proc. Natl. Acad. Sci USA 95: 5818-5823, 1998; Schraudner et al., Plant J. 16: 235-245, 1998; Karpinski et al., Science 284: 654-657, 1999). Thus, modulation of MAPKKK activity, such as ANP activity, in vegetative tissues provides a novel strategy for cross protection from multiple stresses in agriculturally important plants.

Materials and Methods

The above-described results were obtained using the following methods.

Reporter Constructs

The 749 bp soybean GH3 promoter (Hagen et al., Plant Mol. Biol. 17: 567-579, 1991) was fused to a synthetic gene encoding green-fluorescent protein (sGFP) (Chiu et al., Curr. Biol. 6: 325-330, 1996) to visualize the promoter activity. Synthetic ER7 element, TTGTCTCCCAAAGGGAGACAA (SEQ ID NO:______), or mutated ER7, TTGTCTCCCAAAGGGAGAtAA (SEQ ID NO:______) (Ulmasov et al., Science 276: 1865-1868, 1997), was inserted in front of the CaMV 35S minimal promoter (−72) (Sheen, EMBO J. 12: 3497-3505, 1993). The synthetic promoters were fused to a GUS-nos gene to create ER7-GUS and mER7-GUS reporter constructs. Three clones of each construct were tested for auxin induction and gave identical results.

Arabidopsis GST6 (Chen et al., supra), HSP18.2 (Takahashi and Komeda, supra), and RD29A (Yamaguchi-Shinozaki and Shinozaki, supra; Ishitani et al., supra), as well as soybean GH3 (Key, supra; Garbers and Simmons, supra; Walker and Estelle, supra; Leyser, supra) promoters were fused to the luciferase gene to create GST6-LUC, HSP 18.2-LUC, RD29-LUC, and GH3-LUC reporter constructs, respectively.

Effector Constructs

NPK1 and CTR1 were obtained by PCR from tobacco CDNA and an Arabidopsis cDNA library, respectively. NPK1 deletions were generated by PCR. The null NPK1 mutant (K109M) was generated by PCR using the following primers: TACTCGCTATAAtGGAGGTTTCGAT (SEQ ID NO:) and CGCAATCGAAACCTCCaTTATAGCGAGTA (SEQ ID NO:). The mutation was confirmed by DNA sequencing. The PCR products, the coding regions of the constitutively active protein kinases from Arabidopsis (CDPK, APK2, ASK2 (Sheen, Science 274: 1900-1902, 1996), CK1-1 (Klimczal et at., Plant Physiol. 109: 687-696, 1995)), and the coding regions of protein phosphatases (mouse MKP1 (Sun et al., Cell 75: 487-493, 1993), maize PP1 (Smith et al., Plant Physiol. 97: 677-683, 1991), maize PP2A (Sheen, EMBO J. 12: 3497-3505, 1993), and Arabidopsis PP2C (Sheen, Proc. Natl. Acad. Sci. USA 95: 975-980, 1998)) were inserted into a plant expression vector containing the 35SC4PPDK promoter, nos terminator, and DHA tag (Sheen, Science 274: 1900-1902, 1996; Sheen, Proc. Natl. Acad. Sci. USA 95: 975-980, 1998). Three to four independent clones were tested in co-transfection experiments with identical results. Arabidopsis MAPKKKs (ANP1, ANP2, ANP3, and CTR1), MAPKs (AtMPK2 to 7), and serine-threonine protein kinases, ASK1 and CK1-1, were obtained by PCR from an Arabidopsis cDNA library. The kinase-inactive ANP1 mutant (K98M) was generated by PCR using the following primers: TCTCGCCGTCAtgCAGGTTCTGATTGC (SEQ ID NO:______) and GCAATCAGAACCTGcaTGACGGCGAGAAG (SEQ ID NO:______). The mutation was confirmed by DNA sequencing. All PCR products were tagged with two copies of the hemagglutinin (DHA) epitope, and inserted into a plant expression vector containing the 35SC4PPDK promoter and the nos terminator (as described herein). Three to four independent effector clones were tested and gave identical results.

Protoplast Transient Expression

The preparation, electroporation, and incubation of etiolated maize mesophyll protoplasts were as described previously (Sheen, Plant Cell 2: 1027-1038, 1990; Sheen, EMBO J. 12: 3497-3505, 1993). In each electroporation, 2×10⁵ protoplasts were transfected with 30 μg of plasmid DNA carrying a reporter construct alone or with 30 μg of plasmid DNA carrying an effector construct or a vector DNA control. The transfected protoplasts were incubated in medium (5×10⁴/ml) without (− auxin) or with (+ auxin) 1 mM NAA for 14 hours in the dark. GFP fluorescence was observed using UV light as described previously (Sheen et al., Plant J. 8: 777-784, 1995). The GUS (Sheen, Plant Cell 2: 1027-1038, 1990) and luciferase (Sheen, Science 274: 1900-1902, 1996) assays were carried out with cell lysates from 10⁴ protoplasts.

Arabidopsis thaliana, ecotype Bensheim, was grown on B5 medium for 4 weeks. The third pair of leaves were cut into 1.0 mm strips and digested overnight in 1% Cellulase R-10, 0.25% Macerozyme R-10, 0.5 M mannitol, 10 mM CaCl₂, 20 mM KCl, 10 mM MES, pH 5.7, and 0.1% BSA. Protoplasts were released by gentle shaking, filtered through a 75 μm Nylon mesh, collected by centrifugation, and resuspended in W5 solution (Damm et al., Mol. Gen. Genet. 217:6, 1989; Abel and Theologis, supra). Before transfection, protoplasts were resuspended in 0.4 M Mannitol, 15 mM MgCl₂ and 4 mM MES, pH 5.7, to a density of 10⁶ protoplasts/ml. Typically 0.2 ml of the protoplast suspension was mixed with 30 to 50 μg of plasmid DNA containing reporter and effector constructs and equal volume of 40% PEG solution (Damm et al., Mol. Gen. Genet. 217:6, 1989; Abel and Theologis, supra). The transfected protoplasts were diluted with W5 solution, collected by centrifugation, and resuspended in the incubation solution (0.5 M Mannitol, 20 mM KCl, 4 mM MES, pH 5.7).

Determination of Effector Expression

Transfected maize protoplasts (10⁵) were incubated for 5 hours with [³⁵S]-methionine (200 mCi/ml) before harvesting. The NPK1 protein was less stable than other expressed proteins after long incubation (data not shown). Immunoprecipitation with an anti-HA antibody was performed as described previously (Sheen, Science 274: 1900-1902, 1996). The proteins were separated by SDS-PAGE (10%) and visualized by fluorography.

In-Gel Kinase Activity Assay

The transfected protoplasts (10⁵) were incubated for 5 hours before harvesting. The kinase in-gel assay was performed as described previously (Zhang et al., Plant Cell 9: 809-824, 1997).

Immunoprecipitation Kinase Activity Assay Cell lysates from 10⁵ transfected protoplasts were used for immunoprecipitation with an anti-ERK (PAC) antibody (Transduction Laboratory) (Sheen, Science 274: 1900-1902, 1996). The immunoprecipitated proteins were assayed for MAPK activity using MBP as substrate (Bogre et al., Plant Cell 9: 75-83, 1997). The [³²P]-MBP was separated by SDS-PAGE (15%) and visualized by autoradiography.

Protein Kinase and Phosphatase Activity Assays

Cell lysates from 10⁵ transfected protoplasts were used for immunoprecipitation with an anti-HA antibody (Sheen, Science 274: 1900-1902, 1996) to bring down the HA-tagged protein kinases. The immunoprecipitated proteins were assayed using casein as substrate. The [³²P]-casein was separated by SDS-PAGE (10%) and visualized by autoradiography. PP1, PP2A, and PP2C activity assays using transfected maize cell extracts were described previously (Sheen, EMBO J. 12: 3497-3505, 1993; Sheen, Proc. Natl. Acad. Sci. USA 95: 975-980, 1998).

Transgenic Plants

A construct including the 35SC4PPDK promoter (Sheen, EMBO J. 12: 3497-3505, 1993; Sheen, Science 274: 1900-1902, 1996; Sheen, Proc. Natl. Acad. Sci. USA 95: 975-980, 1998), kinase domain of NPK1, DHA tag, and nos terminator was inserted into pART27 binary vector (Gleave, Plant Mol. Biol. 20: 1203-1207, 1992). The resulting plasmid was introduced into Agrobacterium tumefaciens EHA105, and the transformation was performed with Nicotiana tabacum SR1 leaf discs (Chiu et al., Curr. Biol. 6: 325-330, 1996). Several kanamycin-resistant plants were selected from three independent transformation experiments. The kanamycin resistance of T1 progeny plants revealed that the three analyzed independent parental transformants contained more than one copy of the transgene. The seeds were examined under a light microscope. RNA blot and protein blot analyses were performed as described previously (Jang et al., Plant Cell 9: 5-19, 1997).

III. Screening Assays

Efforts to understand plant functional genomics have focused on cDNA sequencing, global gene expression analysis, and generating insertional knockout mutants. The major challenge, however, is to assign functions to the large number of genes provided by plant genomic sequencing projects. To exploit genomic resources in plants, the methods described below enable the skilled worker to identify and elucidate the functions of genes modulating plant signaling cascades using expression systems based on plant protoplast transient assays, including the use of maize and Arabidopsis mesophyll protoplasts. Most traditional bioassays for analyzing plant signaling cascades are performed with whole plants, which are too complex to be used for readily dissecting the underlying cellular, biochemical, and molecular mechanisms. To circumvent the complexity of using intact plants, protoplast transient expression assays facilitate the analysis of genes involved in diverse plant signaling cascades including, without limitation, those modulating hormone (e.g., auxin, cytokinin, ABA, ethylene, jasmonic acid, or brassinosteroids), stress (e.g., cold, heat, salt, osmotic, ethanol, or H₂O₂), metabolite (e.g., glucose, sucrose, sugar analogues, acetate, or other regulatory metabolites), light (e.g., red, far red, blue, or UV light), and pathogen/elicitor signaling pathways.

Identifying regulatory genes involved in signaling pathways is readily accomplished by introducing into one or more protoplasts (1) an expression library and (2) reporter gene construct, and assaying, for example, for the activation or repression of reporter gene expression. The transient nature of the protoplast system allows direct functional analysis of plant genes at an unprecedented high throughput rate and at relatively low cost and short time. The methods described herein are especially useful for unraveling the functions of genes that are difficult to address by traditional genetic and biochemical approaches due to redundancy, lethality, or low levels of expression. In addition, the methods provide for comprehensively assessing virtually all signal transduction molecules important for a plant's response to abiotic and biotic stresses, nutrition signals, and hormonal stimuli that govern plant growth and development. The elucidation and manipulation of signaling cascades in plants will reveal fundamentally important signaling processes, and provide new tools for crop improvement in stress tolerance, disease resistance, and yield enhancement.

In general, the library screening methods of the invention provide a straightforward means for selecting genes of interest from a large library which are further evaluated and condensed to a few active and selective genes. Constituents of this pool are then isolated and evaluated in the methods of the invention to determine their function in a plant signaling cascade. Since the functions of many genes remain unknown, the screening methods of this invention provide novel genes which are active as positive or negative regulators of plant signaling pathways. Therefore, this invention includes such novel genes, as well as the use of both novel and known genes for manipulating signaling pathways in planta.

Expression Libraries

Libraries of cDNA, expressed sequence tags, or genomic sequences are prepared according to standard methods or, if desired, are obtained from publicly available sources. The source of nucleic acid used to construct the expression libraries is not critical to the methods of the invention. For example, cDNA libraries may be generated from a variety of different plant tissues or organs, or from developmentally staged plant material. Expression libraries may also be prepared from nucleic acids isolated from plant material treated with abiotic (e.g., drought, cold, freezing, heat, oxidative stress, herbicide, or UV light) and biotic (e.g., pathogen or elicitor) stresses, or plant hormones (e.g., auxin, cytokinin, ABA, GA, ethylene, brassinosteroids, salicylic acid, or jasmonic acid).

In one example, a cDNA clone bank prepared from poly A+ RNA of leaf material is constructed according to standard methods. The type of cDNA clone bank used in the screening procedure is not believed to be particularly critical. For example, the clone bank may be assembled in a plant expression vector containing the 35SC4PPDK constitutive promoter (or ubiquitin or actin promoters) and the NOS terminator. The TMV omega translation enhancer, if desired, may be incorporated to enhance (approximately 10- to 20-fold) expression. The vector may optionally contain a tag, for example, an HA, MYC, or FLAG epitope tag, at the carboxy-terminus to enable visualization of expressed polypeptides. Once prepared, the cDNA clone bank may be stored in independent pools, each representing about 100-200 individual cDNA clones. Independent pools are subsequently introduced into plant protoplasts for expression analysis.

Reporter Genes

Activation or repression of the transcription of specific reporter genes is used to monitor signaling pathway gene expression. For example, as is demonstrated herein, reporter gene constructs have been used to show that H₂O₂ activates the GST6 and the HSP18.2 promoters, but not the ABA or the drought/cold inducible RD29A promoter in transfected Arabidopsis protoplasts.

To construct new reporters, different promoter regions, including 1-3 kb of 5′ flanking regions and 5′ UTR (or introns and exons and 3′UTR as regulatory sequences), are amplified by standard PCR from genomic DNA and fused to coding regions of a reporter gene. A reporter gene encodes a reporter molecule which is capable of directly or indirectly generating a detectable signal. This includes chromogenic or magnetogenic reporters as well as any light-emitting reporter such as bioluminescent, chemiluminescent, or fluorescent proteins, including but not limited to green fluorescent protein (GFP) and the luciferase (LUC) from the firefly. Any fluorigenic or colorigenic enzymes may also be used which include, but are not limited to, β-galactosidase, alkaline phosphatase, and β-glucuronidase (GUS). Any cell surface antigen may be used, for example, the E. coli thioredoxin-flagellin fusion protein. Preferably, the firefly LUC reporter is used for its sensitivity and easy measurement, whereas the GFP reporter allows direct visualization of the responses in living plant cells.

Because some native promoters contain multiple cis-elements that respond to multiple signals, synthetic promoters that contain a single or limited number of cis-acting signals may also be constructed according to standard methods. For example, a synthetic promoter may be constructed that contains only the ethylene-responsive GCC repeats and the 35S basal TATA promoter that is active in Arabidopsis protoplasts. The simplicity of the protoplast system permits straightforward testing of many potential reporters involved in signaling cascades. The UBQ10-GUS construct may be used as an internal control whose activity is not affected by stress and hormone treatments in Arabidopsis protoplasts.

An assortment of gene promoters, each with its own distinct characteristics embodied in its regulatory sequences are useful for analyzing and identifying regulators of virtually any plant signaling pathway. These include, without limitation, gene promoters that modulate metabolic, stress, hormone, metabolite, light, and plant defense signaling pathways. For example, the GH3 promoter which is inducible and modulated by auxin may be used for evaluating auxin signaling cascades. Other promoters such as HVA1, RD29A, RD22, ARR5, ARR6, and PI2 are useful for identifying regulators of hormone signaling pathways (e.g., abscisic acid (ABA), jasmonate, and cytokinin). Oxidative stress-inducible (H₂O₂) promoters useful in the invention include the GST6, ASN1, and HSP18.2 promoters. Promoters such as RD29A, CBF1, and ASN1 (Asn synthetase) are useful for identifying regulators of osmotic-, salt-, and cold-stress signal pathways. Signaling pathways regulated by heat shock and ethanol are readily assayed using promoters such as HSP18.2, AS1, and GST6. Exemplary plant defense, and pathogen- and elicitor-inducible promoters include GST1, PAL1, PLA2, CHS, PR1, PR2, and PR5 promoters, which may be used to monitor plant defense signaling pathways. Promoters such as CAB, RBCS, CHS, PAL1, NR (nitrate reductase), and ASN1 promoters are useful for identifying regulators of sugar signaling pathways. The CAB, RBCS, C4PPDK, and ASN1 promoters are also useful for analyzing light- and dark-regulated signaling pathways. The CHS promoter may be used to identify regulators of UV light signal transduction pathway. As new promoters are identified in signaling pathway gene expression, they may be directly applied as described herein.

Protoplast Transient Expression Assays

The use of protoplast transient expression assays is suitable for the study of signal responses involving a variety of plant signaling cascades, such as the MAPK cascade described herein. Assays are generally performed three to twelve hours after introduction of DNA into the protoplasts, when protoplasts are most active and exhibit physiological responses. Vital staining with fluorescent diacetate shows that most protoplasts (>95%) are viable for more than seventy-two hours in culture. In addition, stress responses, such as H₂O₂, dark, salt, cold, heat, osmotic, and ABA, are activated in maize and Arabidopsis protoplasts. For any responses that are mediated through calcium as a second messenger, electroporation is to be used for DNA transformation instead of polyethylene glycol/calcium tranformation. The electric shock is transient and does not affect later stress or hormone responses in protoplasts. In addition, if desired, mutant plant lines related to stress, defense, metabolic and hormonal signaling are useful for preparing protoplasts (Ishitani et al., Plant Cell 9: 1935-1949, 1997; Leung and Giraudat, Ann. Rev. Plant Physiol. Plant Mol. Biol. 49: 199-222, 1998; Chang and Shockey, Curr. Opin. Plant Biol. 2: 352-358, 1999; Glazebrook, Curr. Opin. Plant Biol. 2: 280-286, 1999), and are analyzed for the effects of the mutations on a signaling cascade.

Particular examples of suitable plant material useful for preparing protoplasts include, but are not limited to, conifers, petunia, tomato, potato, tobacco, Arabidopsis, lettuce, sunflower, oilseed rape, flax, cotton, sugarbeet, celery, soybean, alfalfa, Medicago, lotus, Vigna, cucumber, carrot, eggplant, cauliflower, horseradish, morning glory, poplar, walnut, apple, grape, asparagus, rice, maize, millet, onion, barley, orchard grass, oat, rye, and wheat. Exemplary methods for preparing maize and Arabidopsis protoplasts for transient assays are provided below.

Etiolated or greening maize mesophyll protoplasts are preferably prepared from maize FR9×FR37, FR992×FR697, or FR27×FRM017 (Illinois Foundation Seed) according to methods described by Sheen (Plant Cell 2:1027-1038, 1990; EMBO J. 12:3497-3505,1993). DNA (approximately 30-100 μg) is introduced into the protoplasts by electroporation that includes the delivery of two 5-10 msec pulses of 300-500 V/cm at 200 μF or higher. If desired, protoplasts are electroporated directly in a 96 well microtiter plate using square wave electroporation conditions (BTX electro square porator T820). Electroporation conditions are generally 1-2 KVolt/cm, 10-50 Usec pulse for 5-20 pulses with approximately 10,000 cells per sample, 5-20 μg DNA in 50-200 μl, in a buffer consisting of 0.5-0.6 M mannitol, 20-75 mM KCl, 1 mM MES or Tris, pH 5.5-8.0.

For preparation and transfection of Arabidopsis protoplasts, Arabidopsis thaliana plants, for example, ecotypes Bensheim, C24, or Col are grown on B5 medium for approximately four weeks. Greenhouse grown (60-80% humidity) plants are exposed to 50-100 μmolm⁻²s⁻¹ of constant light, and plants grown on plates in a growth chamber (40-50% humidity) are exposed to 35-55 μmolm⁻²s⁻¹ of 16 hours to constant light. The second, third, or fourth pair of expanded true leaves of three to four week old plants or cotyledons of 3-6 days old are cut into 1-2 mm strips and digested 3 hours to overnight in 1% cellulase R-10, 0.25% macerozyme R-10, 0.5 M mannitol, 10 mM CaCl₂, 20 mM KCl, 10 mM MES, pH 5.7, and 0.1% BSA. Protoplasts are then released by gentle shaking, filtering through a 75 μm nylon mesh, and subsequently collected by centrifugation, and resuspended in W5 solution (Damm et al., Mol. Gen. Genet. 217:6, 1989). Before transfection, protoplasts are resuspended in 0.4 M mannitol, 15 mM MgCl₂, and 4 mM MES, pH 5.7, to a density of 10⁵-10⁶ protoplasts/ml. The protoplast suspension (0.2 ml) is mixed with approximately 20 to 50 μg of DNA to be introduced into the protoplasts and an equal volume of 40% PEG/Ca⁺⁺ (25-200 mM or Mg⁺⁺) solution (Damm et al., supra), and incubated for five to ten minutes.

Exemplary High Throughput Screening Systems

To identify signaling regulatory genes found in a nucleic acid expression library a number of high throughput assays may be utilized.

For example, to identify genes that are positive regulators of an oxidative stress signaling cascade, Arabidopsis mesophyll protoplasts are co-transfected with the GST6-LUC reporter construct and a pool of clones from a cDNA clone bank in a 96-well microtiter format. Approximately 10² to 10³ protoplasts are placed in each well with 1-20 μg of DNA of the expression plasmids. Relative LUC activity is analyzed according to standard methods after a three to twelve hour incubation period at 23° C. Wells displaying LUC activity are considered as containing a library member that positively regulates gene expression of an oxidative signaling cascade. Positive clone pools are further fractionated and assayed to isolate the library member responsible for the observed effect. To identify negative regulators of oxidative stress signaling, protoplasts are co-transfected with the GST6-LUC reporter and expression library members. Transfected protoplasts are incubated with approximately 50-200 μM H₂O₂, and relative LUC activity is assayed after a three hour incubation at 23° C. Control wells are incubated with the reporter construct and H₂O₂ alone. Since H₂O₂ has been shown to activate expression of the GST6-LUC reporter, wells having decreased LUC activity relative to control wells are taken as containing a library member that expresses a negative regulator of an oxidative stress signaling cascade.

In another working example, positive regulators of a hormone signaling pathway regulated by ABA are identified as follows. Co-expression transient assays are performed by electroporating the HVA1-LUC reporter construct and a pool of expression library clones into approximately 10² to 10³ maize protoplasts. LUC activity is assayed after an appropriate incubation period as described above. Samples positive for LUC activity are identified as having protoplasts that express a positive regulator of an ABA signaling pathway, and the gene encoding the regulator is subsequently isolated from the pool of clones.

Negative regulators of an ABA signaling pathway may also be identified. Co-expression assays are carried out as described above. Transfected protoplasts are further incubated with 1-100 μM ABA, which activates HVA1-LUC reporter gene expression. Samples having decreased LUC activity relative to control samples (protoplasts transfected with reporter construct alone and treated with 1-100 μM ABA) are identified as containing protoplasts that express a gene that negatively regulates an ABA signaling pathway. The gene encoding this regulator is then identified from the pool of clones.

Use

The invention provides for the discovery of regulatory genes in specific signaling pathways including those controlling hormone (such as auxin, cytokinin, ABA, ethylene, jasmonic acid, and brassinosteroids), stress (such as cold, heat, salt, osmotic, ethanol, and H₂O₂), metabolite (such as glucose, sucrose, sugar analogues, acetate or other regulatory metabolites), light (such as red, blue, UV, and far red light), and pathogen/elicitor responses. These key regulatory genes will be used for crop improvement including the manipulation of plant growth and development, control of sink and source balance, yield enhancement, stress tolerance, and eliminating pathogen infection or damage/killing.

All publications and patent applications mentioned in this specification are herein incorporated by reference to the same extent as if each independent publication or patent application was specifically and individually indicated to be incorporated by reference.

What is claimed is:

1. A high throughput assay for rapidly screening a library of nucleic acid molecules to identify a gene product that modulates expression of a gene of interest, said assay comprising:
(a) introducing into one or more plant protoplasts (i) a reporter gene construct operably linked to a promoter of a gene of interest and (ii) a member of a library of nucleic acid molecules, wherein said library member is expressed in said plant protoplasts; and
(b) screening the protoplasts to determine whether the amount of gene expression of said reporter gene construct changes in response to the expression of said library member, a change in gene expression of said reporter gene construct identifying the gene product expressed by said library member as one that modulates expression of the gene of interest.

2. The assay of claim 1, wherein said assay comprises treating said protoplasts with a biotic or an abiotic stress.

3. The assay of claim 1, wherein said assay comprises treating said protoplasts with a hormone.

4. The assay of claim 1, wherein said assay comprises treating said protoplasts with a metabolite.

5. The assay of claim 1, wherein said assay comprises treating said protoplasts with a pathogen, elicitor, or elicitin.

6. The assay of claim 1, wherein said gene of interest expresses a signaling pathway polypeptide.

7. The assay of claim 1, wherein said reporter gene and said library member are introduced into said protoplasts by electroporation or polyethylene glycol transformation.

8. The assay of claim 1, wherein said protoplasts are mesophyll protoplasts.

9. The assay of claim 1, wherein said protoplasts are maize or Arabidopsis protoplasts.

10. The assay of claim 1, wherein said assay comprises at least 10² protoplasts.

11. The assay of claim 1, wherein said assay comprises approximately 10⁴ to 10⁶ protoplasts.

12. The assay of claim 1, wherein said reporter gene expresses a green fluorescent protein, a luciferase, or a β-glucuronidase.

13. The assay of claim 1, wherein said promoter of the gene of interest is an inducible promoter.

14. The assay of claim 13, wherein said inducible promoter is a hormone-, stress-, metabolite-, light-, pathogen-, or elicitor-inducible promoter.

15. The method of claim 14, wherein said promoter of the gene of interest is a repressible promoter.

16. The method of claim 14, wherein said gene of interest expresses a signaling pathway polypeptide.

17. The assay of claim 1, wherein said library of nucleic acid molecules is a genomic, cDNA, expressed sequence tagged, or randomized synthetic library.

18. The assay of claim 1, wherein said library of nucleic acid molecules has at least 10² members.

19. The assay of claim 1, wherein at least ten different members of said library of nucleic acid molecules are introduced into said protoplasts.

20. The assay of claim 1, further comprising determining the sequence of said member of the nucleic acid library.

21. The assay of claim 1, wherein said library member expresses a polypeptide or an RNA molecule that modulates expression of the gene of interest.

22. The assay of claim 1, wherein said library member expresses a signaling pathway polypeptide.

23. The assay of claim 1, wherein said library member expresses a gene product that represses reporter gene expression.

24. The assay of claim 1, wherein said library member expresses a gene product that activates reporter gene expression.

25. The assay of claim 1, wherein said assay takes place in a microtiter well.


SEQUENCE LISTING (27 sequences)

SEQ ID NO: 1 - protein, 274 aa, Arabidopsis thaliana
MANQTQISDKYILGRELGRGEFGITYLCTDRETREALACKSISKRKLRTAVDVEDVRREVTIMSTLPEHPNVVKLKATYEDNETVHLVMELCEGGELFGRIVARGHYTERAAATVARTIAEVVRMCHVNGVMHRDLKPENFLFANKKENSALKAIDFGLSVLFKPGERFTEIVGSPYYMAPEVLKRNYGPEVDVWSAGVILYILLCGVPPFWAETEQGVALAILRGVLDFKRDPWSQISESAKSLVKQMLEPDSTKRLTAQQVLDHPWIQNAKK

SEQ ID NO: 2 - protein, 413 aa, Arabidopsis thaliana
MGNTCVGPSRNGFLQSVSAAMWRPRDGDDSASMSNGDIASEAVSGELRSRLSDEVQNKPPEQVTMPKPGTDVETKDREIRTESKPETLEEISLESKPETKQETKSETKPESKPDPPAKPKKPKHMKRVSSAGLRTESVLQRKTENFKEFYSLGRKLGQGQFGTTFLCVEKTTGKEFACKSIAKRKLLTDEDVEDVRREIQIMHHLAGHPNVISIKGAYEDVVAVHLVMECCAGGELFDRIIQRGHYTERKAAELTRTIVGVVEACHSLGVMHRDLKPENFLFVSKHEDSLLKTIDFGLSMFFKPDDVFTDVVGSPYYVAPEVLRKRYGPEADVWSAGVIVYILLSGVPPFWAETEQGIFEQVLHGDLDFSSDPWPSISESAKDLVRKMLVRDPKKRLTAHQVLCHPWVQVDGV

SEQ ID NO: 3 - protein, 274 aa, Arabidopsis thaliana
MSNQTQISDKYILGRELGRGEFGITYLCTDRETHEALACKSISKRKLRTAVDIEDVRREVAIMSTLPEHPNVVKLKASYEDNENVHLVMELCEGGELFDRIVARGHYTERAAAAVARTIAEVVMMCHSNGVMHRDLKPENFLFANKKENSPLKAIDFGLSVFFKPGDKFTEIVGSPYYMAPEVLKRDYGPGVDVWSAGVIIYILLCGAPPFWAETEQGVALAILRGVLDFKRDPWPQISESAKSLVKQMLDPDPTKRLTAQQVLAHPWIQNAKK

SEQ ID NO: 4 - protein, 280 aa, Arabidopsis thaliana
PSNTVLPYQTPRLRDHYLLGKKLGQGQFGTTYLCTEKSTSANYACKSIPKRKLVCREDYEDVWREIQIMHHLSEHPNVVRIKGTYEDSVFVHIVMEVCEGGELFDRIVSKGHFSEREAVKLIKTILGVVEACHSLGVMHRDLKPENFLFDSPKDDAKLKATDFGLSVFYKPGQYLYDVVGSPYYVAPEVLKKCYGPEIDVWSAGVILYILLSGVPPFWAETESGIFRQILQGKLDFKSDPWPTISEAAKDLIYKMLERSPKKRISAHEALCHPWIVDEQA

SEQ ID NO: 5 - DNA, 1020 bp, Arabidopsis thaliana
  misc_feature (1)...(1020): N = a, t, c or g
gttgtaaaacgacggncagtgaattgtaatacgactcnctatagggcgnaattggagctccaccgcggtggcggccgctctagaactagtggatccatggctaatcaaactcagatcagcgacaagtacatcttaggacgagaactcggtcgcggcgaattcggaatcacgtatctttgtacagatagagagactcgtgaagctttagcttgcaaatcaatctccaagagaaagctccgaaccgccgtcgatgtggaagacgtccgtcgtgaagtcacgatcatgtcaactttaccggaacacccaaacgttgtgaaacttaaagcgacttatgaggataacgagaccgtgcatcttgtgatggagctttgtgaaggaggtgagctttttggtcggattgttgcaagaggacattatacagagcgtgcggcggctaccgtcgcgagaacgatcgcggaagttgtgaggatgtgtcatgtcaatggtgttatgcatagagatttgaagcctgagaatttcttgtttgctaacaagaaggagaattctgcacttaaggctattgattttggtttatctgttctctttaaacctggagagaggtttacagagattgttggaagtccttattatatggctccagaagtgttgaagagaaattatggaccagaggttgatgtgtggagtgctggagttatcctctacatcttgctttgtggtgttcctccgttttgggcagagactgaacaaggtgtggctcttgccatcttgaggggagttcttgattttaagagagatccttggtcgcagatatcagagagcgcaaagagccttgtgaagcagatgttggaacctgattcaactaagcgtttgactgctcagcaagttcttgatcacccttggatacagaatgcaaagaaaaggatcaagcttatcgataccgtcgacctcgagggggggcccggtaccagctttngttccctttagtgagggttaatttcgagcttggcgtaatcatgtcat

SEQ ID NO: 6 - DNA, 20 bp, Hordeum vulgare
tccaccgagatgccgacgca

SEQ ID NO: 7 - DNA, 26 bp, Hordeum vulgare
gttggaggccatggtcgtctcacgat

SEQ ID NO: 8 - DNA, 29 bp, Arabidopsis thaliana
gaagatctatgggtaatacttgtgttgga

SEQ ID NO: 9 - DNA, 28 bp, Arabidopsis thaliana
gtcaaggcctgtcgacttgaacccatgg

SEQ ID NO: 10 - DNA, 33 bp, Arabidopsis thaliana
gcggatccatggctaatcaaactcagatcagcg

SEQ ID NO: 11 - DNA, 29 bp, Arabidopsis thaliana
gtcaaggcctcatcagtgagaacatgttc

SEQ ID NO: 12 - DNA, 30 bp, Arabidopsis thaliana
gcggatccatggagacgaagccaaacccta

SEQ ID NO: 13 - DNA, 30 bp, Arabidopsis thaliana
gtcaaggccttgcttgttcatcgacaatcc

SEQ ID NO: 14 - DNA, 30 bp, Arabidopsis thaliana
catgccatggctccggcgactaattcaccg

SEQ ID NO: 15 - DNA, 32 bp, Arabidopsis thaliana
gtcaaggcctattcttcaagaaccatttatcg

SEQ ID NO: 16 - DNA, 30 bp, Arabidopsis thaliana
gcggatccatggctcgagctccggtgacca

SEQ ID NO: 17 - DNA, 31 bp, Arabidopsis thaliana
gtcaaggcctattcttcaagaaccaactatg

SEQ ID NO: 18 - DNA, 33 bp, Arabidopsis thaliana
gcggatccatggctaagtcagagctggtgaaag

SEQ ID NO: 19 - DNA, 31 bp, Arabidopsis thaliana
gtcaaggcctattctttaggaaccatgaatg

SEQ ID NO: 20 - DNA, 33 bp, Arabidopsis thaliana
gcggatccatggctaagtatgacgttgtcaagg

SEQ ID NO: 21 - DNA, 28 bp, Arabidopsis thaliana
gtcaaggcctattcttcaagtaccacgg

SEQ ID NO: 22 - DNA, 21 bp, Artificial Sequence
  Oligonucleotide primer
ttgtctcccaaagggagacaa

SEQ ID NO: 23 - DNA, 21 bp, Artificial Sequence
  Oligonucleotide primer
ttgtctcccaaagggagataa

SEQ ID NO: 24 - DNA, 25 bp, Artificial Sequence
  Oligonucleotide primer
tactcgctataatggaggtttcgat

SEQ ID NO: 25 - DNA, 29 bp, Artificial Sequence
  Oligonucleotide primer
cgcaatcgaaacctccattatagcgagta

SEQ ID NO: 26 - DNA, 27 bp, Artificial Sequence
  Oligonucleotide primer
tctcgccgtcatgcaggttctgattgc

SEQ ID NO: 27 - DNA, 29 bp, Artificial Sequence
  Oligonucleotide primer
gcaatcagaacctgcatgacggcgagaag